(19) United States
(12) Patent Application Publication
(10) Pub. No.: US 2008/0069889 A1
(43) Pub. Date: Mar. 20, 2008

(54) Compressible resilient granules and formulations prepared therefrom

(76) Inventor: S. Rao Cherukuri, Vienna, VA (US)

Correspondence Address: RICHARD R. MUCCINO, 758 SPRINGFIELD AVENUE, SUMMIT, NJ, 07901, US

(21) Appl. No.: 11/899,601

(22) Filed: Sep. 5, 2007

Related U.S. Application Data
(63) Continuation-in-part of application No. 11/715,821, filed on Mar. 7, 2007.
(60) Provisional application No. 60/780,304, filed on Mar. 7, 2006.

(51) Int. Cl. A61K 9/14; A61K 31/715; A61K 9/50; A61P 43/00

(52) U.S. Cl. 424/493; 424/499; 514/54

(57) ABSTRACT

The present invention provides resilient self-adhering granules which comprise a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m². The present invention also provides oral dosage compositions comprising the resilient self-adhering granules and methods for making and using the resilient self-adhering granules.

### PRIORITY DATA

This continuation-in-part application claims priority from U.S. patent application Ser. No. 11/715,821, filed on 7 Mar. 2007, which in turn claims priority from U.S. provisional patent application Ser. No. 60/780,304, filed on 7 Mar. 2006.

### FIELD OF THE INVENTION

The present invention provides resilient self-adhering granules which comprise a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m². The present invention also provides oral dosage compositions comprising the resilient self-adhering granules and methods for making and using the resilient self-adhering granules.

### BACKGROUND OF THE INVENTION

Pharmaceuticals present in oral dosage forms such as tablets, caplets, capsules, sachets, powders, etc. can have a number of problems. When such forms are administered at high doses, the oral dose form can be rather large due to the drug, carrier, and other excipients needed. Such forms are commonly referred to as “horse-pills.” Many patients refuse to take such “horse-pills” and try to cut or crush them into smaller sizes to facilitate easier swallowing. However, in the process, the tablets generally break and do not provide uniformity in dosing, or because they lack the protective coating in the broken areas, the patient experiences the bitter taste of a typical active agent. Additionally, such breaking can result in loss of the drug since typical drug compositions easily disintegrate into powders or particles that cannot be easily recovered. Accordingly, materials and methods for providing oral dosage forms that can be easily modified and avoid the above-mentioned problems continued to be sought through on-going research and development efforts.

Some products require the making of microparticulate dispersions or coated beads for special delivery and are formed into a capsule or tablet. Tablets usually are not flexible and coated particles and beads are often crushed causing the tablet to loose its properties. Accordingly, flexible resilient granules are required to minimize or eliminate any change of properties in processing to protect the product. Resilient granules also eliminate the need for capsule dosage form, by converting microparticulate particles and beads into flexible tablets with good cushioning properties and less abrasiveness.

### SUMMARY OF THE INVENTION

The present invention provides resilient self-adhering granules which comprise a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m².

The present invention also provides oral dosage compositions comprising resilient self-adhering granules which comprise a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m² and the granules have agglomerated to form the oral dosage composition.

The present invention further provides methods for making a resilient self-adhering granule which comprises a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m², which comprises the steps of:
(a) mixing and heating the polysaccharide and binder in a mixer to form a reaction mixture; and
(b) extruding the reaction mixture from step (a), cooling the reaction mixture to room temperature, milling the reaction mixture to a particular granule size, and cooling the reaction mixture in a freezer.

The present invention still further provides methods for administering resilient self-adhering granules to a subject comprising:

a) providing resilient self-adhering granules which comprises a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m²; and

b) administering the oral dosage composition to a subject's oral cavity, wherein the majority of the resilient self-adhering granules is released in the gastrointestinal tract.

### DETAILED DESCRIPTION OF THE INVENTION

The present invention provides resilient self-adhering granules which comprise a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m². The present invention also provides oral dosage compositions comprising the resilient self-adhering granules and methods for making and using the resilient self-adhering granules.

As used herein, the following terms have the given meanings:

The terms “a,” “an,” and, “the” include plural references unless the context clearly dictates otherwise. Thus, for example, reference to “a drug” includes reference to one or more of such drugs, and reference to “an excipient” includes reference to one or more of such excipients.

The term “active agent,” “bioactive agent,” “drug”, “pharmaceutically active agent,” and “pharmaceutical,” may be used interchangeably to refer to an agent or substance that has measurable specified or selected physiologic activity when administered to a subject in a significant or effective amount.

The term “about” is used to provide flexibility to a numerical range endpoint by providing that a given value may be “a little above” or “a little below” the endpoint.

The term “admixed” means that the drug and/or other ingredients can be dissolved, dispersed, or suspended in the carrier. In some cases, the drug may be uniformly admixed in the carrier.

The term “agglomeration” refers to a cluster of like particles in which the particles are held together by surface forces. Agglomeration is the aggregation or massing of particles together as in a clump.

The term “comestible” refers to the absorption, uptake, or digestion of an active agent in the gastrointestinal tract at a point past the mouth. Generally, this term is meant to exclude traditional gums and chews.

The terms “concentrations”, “amounts”, and other “numerical data” may be expressed or presented herein in a range format. Such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The terms “formulation” and “composition” may be used interchangeably and refer to a mixture of two or more compounds, elements, or molecules. In some embodiments the terms “formulation” and “composition” may be used to refer to a mixture of one or more active agents with a carrier or other excipients.

The term “free-flowing” refers to the ability to not agglomerate under atmospheric pressure or otherwise to not substantially adhere to objects of a different material.

The term “low-pressure, reversible agglomeration” refers to granules that agglomerate with low pressure, typically at or below 6,500 kilonewtons/m² and that yet retain their individuality to a degree sufficient to allow easy delamination of particles from the agglomerate, which are substantially intact with their original individual properties. In some cases, such granules may not substantially rupture or break upon delamination from the agglomerate.

The term “non-resilient” refers to materials that do not retain many or most of their individual characteristics, such as internal structure, crystallinity, melting point, etc., when acted upon by mild to moderate external pressures. Non-resilient materials, such as compressible granules, are used to form traditional tablets and do not agglomerate but rather typically disintegrate into smaller particles or powder when subjected to light pressure.

The term “pharmaceutically acceptable carrier” and “carrier” may be used interchangeably, and refer to any inert and pharmaceutically acceptable material, carrier, or excipient that has substantially no biological activity, and makes up a substantial part of the formulation.

The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide. Chemical modification of a pharmaceutical compound (i.e., drug) into a salt is a technique well known to pharmaceutical chemists to obtain improved physical and chemical stability, hygroscopicity, and solubility of compounds. See, e.g., H. Ansel et. al., Pharmaceutical Dosage Forms and Drug Delivery Systems (6^{th} Ed. 1995) at pp. 196 and 1456-1457.

The terms “plurality of items”, “structural elements”, “compositional elements”, and “materials” may be presented in a common list for convenience. However, these lists should be construed as though each member of the list is individually identified as a separate and unique member. Thus, no individual member of such list should be construed as a de facto equivalent of any other member of the same list solely based on their presentation in acommon group without indications to the contrary.

The term “prodrug” refers to compounds, which undergo biotransformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

The term “resilient” refers to the ability of a material to retain its general characteristics and/or individuality even though acted upon by an outside force. For example, granules that are resilient may retain many or most of their individual characteristics, such as internal structure, crystallinity, melting point, etc., even when acted upon by mild to moderate external pressures. A resilient material is capable of withstanding shock without permanent deformation or rupture, tending to recover from or easily adjust from change. Resilient granules, such as those of the present invention, may be pressed between a thumb and index finger to agglomerate to form a resilient mass.

The term “self-adhering” refers to a granule's ability to agglomerate with other like granules or particles under various pressures.

The term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. A composition that is “substantially free of” an ingredient or element may still actually contain such item as long as there is no measurable effect thereof.

The term “therapeutically effective amount” means an amount of a therapeutically effective compound, or a pharmaceutically acceptable salt thereof, which is effective to treat, prevent, alleviate or ameliorate symptoms of a disease.

The present invention provides resilient, self-adhering granules that are capable of low-pressure, reversible agglomeration. For example, when the present granules are pressed between one's thumb and index finger, the granules agglomerate to form a resilient mass, which does not substantially stick to the fingers but rolls freely and can assume any shape desired. Therefore, the granules can be termed free flowing as well as resilient and compressible. This is to be contrasted with compressible granules, which are used to form traditional tablets or traditional chewable tablets or orally disintegrating tablets. For example, compressible granules do not agglomerate but rather typically disintegrate into smaller particles or powder when subjected to light pressure.

Because of the unique characteristics of resiliency and agglomeration under light pressure, dosage forms such as tablets and caplets made from the present granules are resistant to breakage or chipping. Further, if a patient breaks the tablet or caplet either intentionally to reduce dosage size or by accident, the dosage form can be easily reconstructed to its original shape and mass without any substantial loss of material and with minimal visibly distinct breakage mark. The tablet or caplet can be easily reconstructed to its original shape simply by holding the pieces opposite to each other and by applying light pressure in opposite directions. The pieces simply bond or agglomerate together. This may be contrasted with dosage forms made from art-known granules wherein the dosage form cannot be reconstructed ordinarily under light pressure.

For example, a traditional art-known tablet with 500 mg or more of active ingredient when formulated with all needed excipients and can become unwieldy to a patient. The present invention provides several advantages over such a dosage form. Because of the compactness achieved by virtue of the unique internal structure of the resilient, self-adhering granules, the oral dosage forms produced do not become so big to be considered “horse-pill”. Even if the dosage form is considered big for some patients, the breaking of the tablet or caplet is much more easily accomplished compared with traditional tablets. Since the granules have a relatively high moisture content, the tablet portions can easily and quickly glide in the patient's mouth with minimal residence time, which minimizes unpleasant taste of uncoated active drug. To further improve this embodiment, the active drug can be taste-masked or coated. While the art-known granules may comprise coated or taste-masked active drug because such granules lack the unique internal structure of the presently disclosed granules, the resulting product is not either compact, or difficult to break, or does not provide uniformity of dosing, or does not glide in the mouth as comfortably, or has a longer residence time in the mouth. In addition, because the art-known granules are not resilient, the art-known granules, when presented to a patient in a rapidly disintegrating composition, brings out a powdery gritty mouth-feel to the patient. In contrast, the granules of the present invention create a smooth, pleasant mouth-feel. This is especially advantageous for pediatric or geriatric patients or patients who have a compromised saliva production (e.g., xerostemia patients or AIDS patients).

The present invention further provides a mixture of the present resilient granules and non-resilient granules in which the resilient granules comprise an active agent and the non-resilient granules comprise an inactive agent, or vice versa, that is a mixture of the present resilient granules and non-resilient granules in which the resilient granules comprise an inactive agent and the non-resilient granules comprise an active agent.

The present invention still further provides a mixture of the present resilient granules and non-resilient granules in which the resilient granules comprise an active agent in immediate release form and the non-resilient granules comprise the same active agent but in delayed release form, or vice versa, that is a mixture of the present resilient granules and non-resilient granules in which the resilient granules comprise an active agent in delayed release form and the non-resilient granules comprise the same active agent but in immediate release form. Delayed release forms of active agents are well known in the art.

The present invention yet further provides a mixture of the present resilient granules and non-resilient granules in which the resilient granules comprise a first active agent and the non-resilient granules comprise a second active agent. In this latter embodiment, the first and second active agents can both be in immediate release form, can both be in delayed release form, or one active agent can be in immediate release form and the other active agent can be in delayed release form.

The present invention yet further provides a mixture of the present resilient granules in which a first portion of the resilient granules comprises an active agent and a second portion of the resilient granules does not comprise an active agent. The present invention yet further provides a mixture of the present resilient granules in which a first portion of the resilient granules comprises an active agent in immediate release form and a second portion of the resilient granules comprises the same active agent but in delayed release form. The present invention yet further provides a mixture of the present resilient granules in which a first portion of the resilient granules comprises a first active agent and a second portion of the resilient granules comprises a second active agent. In this latter embodiment, the first and second active agents can both be in immediate release form, can both be in delayed release form, or one active agent can be in immediate release form and the other active agent can be in delayed release form.

Polysaccharides are polymers made up of many monosaccharides joined together by glycosidic linkages. Generally, they are very large, often branched, molecules. They tend to be amorphous, insoluble in water, and have no sweet taste. Examples of polysaccharides include starch, glycogen, cellulose, and chitin. Polysaccharides have a general formula of Cₙ(H₂O)ₙ₋₁ where n is usually a large number between 200 and 2500. The general formula can also be represented as (C₆H₁₀O₅) n where n=40−3000. Polysaccharides include, but are not limited to, simple sugars, complex sugars, fibers, starches, pectins, dextrans, dextrins, natural gums, synthetic gums, mucilages, derivatives thereof, components thereof, and mixtures thereof.

Basic carbohydrate units are called monosaccharides, e.g., glucose, galactose, and fructose. The general chemical formula of an unmodified monosaccharide is (C.H₂O)ₙ, where n is any number of three or greater. Monosaccharides can be linked together in almost limitless ways. Two joined monosaccharides are called disaccharides, e.g., sucrose and lactose. Carbohydrates containing between about three to six monosaccharide units are termed oligosaccharides; anything larger than this is a polysaccharide. Polysaccharides, such as starch, glycogen, or cellulose, can reach many thousands of units in length.

Carbohydrates are molecules having straight-chain aldehydes or ketones with many hydroxyl groups added, usually one on each carbon atom that is not part of the aldehyde or ketone functional group. Carbohydrates include monosaccharides, disaccharides, oligosaccharides and polysaccharides.

The polysaccharides can include, but are not limited to, simple sugars, complex sugars, fibers, starches, pectins, dextrans, dextrins, natural gums, synthetic gums, mucilages, derivatives thereof, components thereof, and mixtures thereof. In a preferred embodiment, the polysaccharide is a dextrin. In a more preferred embodiment, the dextrin is a maltodextrin.

Dextrins are a group of low-molecular-weight carbohydrates produced by the hydrolysis of starch. Dextrins are mixtures of linear α-(1, 4)-linked D-glucose polymers. They have the same general formula as carbohydrates but are of shorter chain length. Dextrins are water-soluble, white to slightly yellow solids, which are optically active. The cyclical dextrins are known as cyclodextrins. They are formed by enzymatic degradation of starch by certain bacteria, for example Bacillus macerans. Cyclodextrins have toroidal structures formed by 6-8 glucose residues.

Maltodextrins are partially hydrolysated starches and are moderately sweet polysaccharide. Maltodextrins are defined by the FDA as products having a dextrose equivalent (DE) less than 20. Starch hydrolysates are generally produced by heat, acid, or enzymes. This process breaks down the starch and converts some of the starch to dextrose. With adjustments, this process yields more or less dextrose. Maltodextrins are therefore classified by dextrose equivalence. Dextrose equivalents are a measure of the reducing sugars present calculated as dextrose and expressed as a percentage of the total dry substance. Maltodextrins can have a dextrose equivalent of up to 20. At above 20 DE, the product is then generally classified as corn syrup solids, which are completely soluble and impart significant sweetness.

Dextran is a complex, branched polysaccharide made of many glucose molecules joined into chains of varying lengths, used as an antithrombotic (anti-platelet), and to reduce blood viscosity. The straight chain consists of α (1->6) glycosidic linkages between glucose molecules, while branches begin from α (1>3) linkages, and in some cases, α (1->2) and α (1->4) linkages as well.

The binders that may be used in the present invention include: syrups such as maltitol syrups of varying viscosities, emulsifiers that can function as binders, fats and waxes that can function as binders, and gums that function as binders. Examples of emulsifiers that function as binders include, but are not limited to, acetylated mono, di, or triglycerides or other esters; or polyethyleneglycol esters. Additionally, plasticizers may also be used. Examples of fats and waxes that function as binders include, but are not limited to, bee's wax, carnuba wax, spermaceti, etc. Synthetic waxes include, but are not limited to, mineral oil, paraffin, microcrystalline wax, and polyethylene wax. Examples of gums that function as binders include, but are not limited to, gum Arabica, gum tragacanth, gum acacia, and fiber gums. In one embodiment, the binder can be a maltitol syrup.

The resilient, self-adhering granules may further comprise a sugar alcohol. Sugar alcohols are hydrogenated forms of carbohydrates, whose carbonyl group (aldehyde or ketone, reducing sugar) has been reduced to a primary or secondary hydroxyl group. This term is also commonly known as polyol, polyhydric alcohol, or polyalcohol. Sugar alcohols include, but are not limited to, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, and mixtures thereof. In a preferred embodiment, the sugar alcohol can be maltitol.

The resilient, self-adhering granules can be pharmaceutically acceptable. Such pharmaceutically acceptable granules may be inert (i.e., comprise no pharmaceutically active agent) or may comprise one or more pharmaceutically active agents. Pharmaceutical oral dosage forms such as tablets, caplets, capsules, sachets, powders and the like comprising compressible resilient granules of the present invention are also provided. Methods for making and using such granules and dosage forms are also provided.

The resilient, self-adhering granules of the present invention can be compressed into a tablet or a caplet of a desirable shape and size. Such compression can be achieved by using compression forces that are known in the pharmaceutical industry, such as those forces ranging from about 100 to about 4000 lbs/in². However, the present granule can also agglomerate under low-pressure. Generally, such low pressure can be less than 6,500 kilonewtons/m². Additionally, the granules of the present invention can withstand pressures of up to 40 kilonewtons/m² without losing their resiliency.

The resilient, self-adhering granules of the present invention can have a moisture content ranging from about 0.1% to about 10%. In one embodiment, the moisture content of the granule may range from about 0.1% to about 6%, preferably from about 0.5% to about 4%, and more preferably from about 1% to about 4%. In another embodiment, the moisture content of the granule may range from about 1% to about 8%, preferably from about 0.5% to about 7%, and more preferably from about 3% to about 6%. This moisture content is generally considered to be high for a compressible tablet.

Water activity or a_{w} is the energy state of water in a substance. It is defined as the vapor pressure of water divided by that of pure water at the same temperature; therefore, pure distilled water has a water activity of exactly one. In one embodiment, the present granules can have a water activity less than about 0.6. Alternatively, the water activity may be less than about 0.5, less than about 0.4, less than about 0.3, or less than about 0.2. In another embodiment, the water activity may range from about 0.1 to about 0.5, from about 0.1 to about 0.4, from about 0.1 to about 0.3, from about 0.1 to about 0.2, or from about 0.15 to about 0.2.

The compressible granules of the present invention can be compressed into a tablet or a caplet of desired shape and size. The tablets and caplets thus formed can be, for example, round, oval, square, rectangular, cylindrical, oblong, triangular, octagonal, hexagonal, and the like. In addition, the tablets and caplets may be scored to provide dosing flexibility. For example, the tablets can be scored to permit two half dosings, or three one-third dosings (i.e., scored twice), or four one-quarter dosings. Other shapes and scoring configurations are also feasible.

The unique internal structure of the present granules can permit dosage forms such as tablets or caplets that can be compact, i.e., have a high drug loading per surface area. Because of this unique characteristic of compactness, the present granules can be used to prepare oral dosage forms such as tablets or caplets that carry a high drug loading and yet permit easy swallowing without creating the fear of choking. The compactness of the present dosage forms may be measured as a function of drug loading versus surface area, i.e., mg/cm². In one embodiment, the compactness can be expressed as 0.05, 0.08, 0.1, 0.2, 0.3, 0.4, 0.5, 0.6, or 0.7 in mg/cm². In another embodiment, the compactness of present granules may be expressed as ranging from 0.01 to 0.5, 0.01 to 0.1, 0.01 to 0.2, or 0.01 to 0.3, in mg/cm². In another embodiment, the compactness may range from 0.05 to 0.1, 0.05 to 0.2, 0.05 to 0.3, and 0.05 to 0.4, in mg/cm². In another embodiment, the compactness may range from 0.1 to 0.2, 0.1 to 0.3, 0.1 to 0.4, or 0.1 to 0.5, in mg/cm².

In addition to the above characteristics, the compressible granules of the present invention, despite its high moisture content, can be free-flowing and may not substantially stick to metal surfaces such as punches and dies and processing equipment such as granulators, dryers, mixers, coaters, etc. This attribute can permit high volume manufacture of tablets, caplets, capsules, powders, sachets, etc., offering significant commercial advantage. It should be mentioned that it is the prevailing understanding in the industry that high moisture content in granules causes several difficulties in pharmaceutical formulations, and is thus actively discouraged. For example, granules are traditionally dried to have a moisture content of 0.1% or even much less to facilitate free-flow of granules, which is an essential requirement for achieving high-speed high-volume tabletting to meet commercial production demands.

Further, it is generally considered that high moisture content is detrimental to acceptable stability of the formulations. However, the present invention has uniquely provided stable, free flowing compressible granules that are highly desirable both from a consumer point-of-view and from industry point-of-view.

Viscosity is a measure of the resistance of a fluid to deform under shear stress. It is commonly perceived as “thickness”, or resistance to flow. Viscosity describes a fluid's internal resistance to flow and may be thought of as a measure of fluid friction. Thus, water is “thin”, having a lower viscosity, while vegetable oil is “thick” having a higher viscosity. When looking at a value for viscosity the number that one most often sees is the coefficient of viscosity, simply put this is the ratio between the pressure exerted on the surface of a fluid, in the lateral or horizontal direction, to the change in velocity of the fluid as you move down in the fluid (this is what is referred to as a velocity gradient). For example, at “room temperature”, water has a nominal viscosity of 1.0×10⁻³ Pa·s and motor oil has a nominal apparent viscosity of 250×10-³ Pa·s.

In one embodiment, the resilient, self-adhering granules of the present invention can comprise a binder or a mixture of binders wherein at least one of the binders can have a viscosity of at least about 5,000 millipascals (mPa·s). In some embodiments, the viscosity of the binder can be greater than about 6,000 milliPascals. In yet another embodiment, the viscosity of the binder can be greater than about 7,000 milliPascals, about 8,000 milliPascals, about 10,000 millipascals, about 12,000 millipascals, about 14,000 milliPascals, about 16,000 milliPascals, about 18,000 milliPascals, about 20,000 millipascals, about 22,000 milliPascals, about 24,000 millipascals, about 26,000 millipascals, about 28,000 millipascals, about 30,000 milliPascals, about 60,000 milliPascals, about 80,000 milliPascals, about 100,000 milliPascals, about 120,000 milliPascals, about 150,000 milliPascals, about 180,000 milliPascals, about 210,000 milliPascals, and about 240,000 milliPascals.

In some embodiments, the viscosity of at least one of the binders can range from about 5,000 milliPascals to about 250,000 millipascals, from about 5,000 milliPascals to about 200,000 millipascals, from about 5,000 millipascals to about 180,000 millipascals, from about 5,000 milliPascals to about 150,000 milliPascals, from about 5,000 milliPascals to about 130,000 millipascals, from about 5,000 milliPascals to about 100,000 milliPascals, from about 5,000 milliPascals to about 80,000 milliPascals, from about 5,000 millipascals to about 60,000 milliPascals, from about 5000 millipascals to about 50,000 millipascals, from about 5000 millipascals to about 40,000 millipascals, from about 5000 millipascals to about 30,000 milliPascals, from about 5,000 millipascals to about 25,000 milliPascals, from about 5,000 milliPascals to about 20,000 milliPascals, from about 5000 millipascals to about 15,000 milliPascals, and from about 5,000 millipascals to about 10,000 milliPascals.

In some embodiments, the viscosity of at least one of the binders can range from about 7,000 milliPascals to about 30,000 milliPascals, from about 7,000 milliPascals to about 25,000 milliPascals, from about 7,000 milliPascals to about 20,000 milliPascals, from about 7,000 milliPascals to about 15,000 millipascals, from about 7,000 milliPascals to about 13,000 milliPascals, and from about 7,000 millipascals to about 10,000 millipascals, from about 7,000 millipascals to about 250,000 millipascals, from about 7,000 milliPascals to about 200,000 milliPascals, from about 7,000 millipascals to about 180,000 milliPascals, from about 7,000 millipascals to about 150,000 millipascals, from about 7,000 milliPascals to about 130,000 milliPascals, and from about 7,000 milliPascals to about 100,000 milliPascals, from about 7,000 milliPascals to about 80,000 milliPascals, from about 7,000 milliPascals to about 70,000 millipascals, from about 7,000 millipascals to about 60,000 millipascals, from about 7,000 milliPascals to about 50,000 millipascals, and from about 7,000 milliPascals to about 40,000 milliPascals.

In some embodiments, the viscosity of at least one of the binders can range from about 10,000 millipascals to about 30,000 milliPascals, from about 10,000 millipascals to about 25,000 milliPascals, from about 10,000 milliPascals to about 20,000 millipascals, from about 10,000 milliPascals to about 15,000 milliPascals, and from about 10,000 milliPascals to about 13,000 milliPascals, from about 10,000 milliPascals to about 250,000 milliPascals, from about 10,000 milliPascals to about 200,000 milliPascals, from about 10,000 millipascals to about 180,000 milliPascals, from about 10,000 millipascals to about 150,000 milliPascals, from about 10,000 millipascals to about 120,000 milliPascals, from about 10,000 milliPascals to about 100,000 milliPascals, from about 10,000 millipascals to about 80,000 millipascals, from about 10,000 millipascals to about 70,000 millipascals, from about 10,000 milliPascals to about 50,000 millipascals, and from about 10,000 milliPascals to about 40,000 millipascals.

In some embodiments, the viscosity of at least one of the binders can range from about 8,000 millipascals to about 250,000 millipascals, from about 8,000 millipascals to about 200,000 milliPascals, from about 8,000 milliPascals to about 150,000 milliPascals, from about 8,000 milliPascals to about 100,000 milliPascals, from about 8,000 millipascals to about 80,000 millipascals, and from about 8,000 milliPascals to about 50,000 millipascals.

In some embodiments, the viscosity of at least one of the binders can range from about 10,000 millipascals to about 28,000 milliPascals, from about 10,000 milliPascals to about 23,000 millipascals, from about 10,000 millipascals to about 18,000 milliPascals, from about 10,000 milliPascals to about 14,000 milliPascals, and from about 10,000 milliPascals to about 13,000 milliPascals.

A mixture of binders may also be used in the present invention such that the binders in combination may possess a viscosity as recited in the previous paragraphs. For example, a binder such as polysaccharide syrups—Lycasin (maltitol syrup), corn syrup, gum solutions and cellulose solutions of a lower viscosity (e.g., 2,000 millipascals) may be combined in different proportions with another binder of higher viscosity (e.g., Lycasin HBC, maltitol syrup of 13,000 milliPascals or Lycasin HDS maltitol syrup with a viscosity of about 250,000 millipascals) to provide an overall viscosity for the binder mixture of at least 5,000 millipascals, or other viscosities recited above. One of ordinary skill in the art is expected to be quite familiar with the arithmetic calculations necessary to arrive at the amounts of each contributing binder.

It is to be further noted that the viscosities stated herein are those stated in the literature for the particular product as mentioned by the manufacturer of the product or in the reference books such as Handbook of Excipients or other equivalent source. In some cases, the viscosities are measured at a certain temperature, for example at 20° C. or 30° C. One of skill in the art would readily note that viscosity is often a function of temperature over a certain range of temperatures for most products. For example, Lycasin HDS is a maltitol syrup manufactured by Roquette America, Inc. The manufacturer states that Lycasin HDS has a viscosity of about 250,000 milliPascals at 30° C. The viscosity for this product cannot be measured at temperatures below 30° C., without some extraordinary effort. Therefore, this is the viscosity that is being used in this application for this particular product. Since one of skill in the art would appreciate this technical aspect, other products listed in the present application have been similarly characterized as necessary, which one skilled in the art would recognize.

The compressible resilient granules of the present invention may be inert, i.e., do not comprise a pharmaceutically active agent, or may comprise one or more pharmaceutically active agents. The active agent may be present homogenously throughout the granule or coated on the surface of the granule. In any case, the granules may be used to prepare oral dosage forms such as tablets, caplets, capsules, sachets, powders and the like. For example, where the compressible resilient granules are inert, the granules may be used to provide sufficient flexibility for other ingredients in the dosage form, including other granules, whether they are known granules that are not resilient or whether they are resilient granules of this invention, so that such ingredients can be compressed into a tablet or caplet or such ingredients can be compressed into a mass of material that can be filled into a capsule.

In one embodiment, the resilient, self-adhering granules of the invention can be inert and mixed with known non-resilient granules which can comprise a pharmaceutically active agent and, for example, coated with a taste-masking coating material or a modified release coating material. These two types of granules (resilient and non-resilient) can be compressed into a dosage form such as a tablet or a caplet where there is no substantial breakage or loss of coating on the non-resilient granules. This is a significant advancement in the pharmaceutical arts because the problem of breakage of the coating of coated granules during compression is a well-known problem in the art. For example, enteric coated granules are not generally compressed for the fear that the enteric coating will break under the compression forces generally employed in pharmaceutical tabletting, and thus, cause premature leakage of the active drug in the stomach, before the drug reaches its intended target, the intestines. Similarly, taste-masked granules are generally known to rupture during compression, causing the drug to dissolve in the mouth while swallowing and thus creating a bitter unpleasant taste for the patient. The present invention provides oral dosage compositions that provide an active agent to a subject in the gastrointestinal tract at a point after the mouth. As such, the compositions and methods of the present method can be considered comestible as defined herein.

In another embodiment, the non-resilient known granules and the resilient granules of the present invention may both comprise a pharmaceutically active agent. In some embodiments, the active agent can be the same or in some embodiments, the active agent can be different in each granule type. Further, the dosage of the active agent in each case may be the same or different. For example, where the active agent is the same in both the resilient and non-resilient granules, the non-resilient granules may be coated to provide modified release for the active agent while the resilient granules of the invention provide an immediate delivery of the active agent. In another embodiment, different active agents may be present in a single granule or individually present in individual granules. Also, a combination of these granules may be present as immediate release, controlled release, or mixtures thereof. A variety of dosage and release characteristics can be obtained by following the concepts and examples presented herein.

The unique characteristics of the present granules permit incorporation of a wide variety of active ingredients, regardless of their aqueous solubility or particle size. For example, highly water-soluble, sparingly water-soluble, and water-insoluble actives can be employed. Particle sizes of the actives could range from about 50 nm to about 500 μM. Thus, nanoparticles and microparticles can be used to make the present resilient, self-adhering granules.

The resilient self-adhering granules of the present invention may further comprise an active agent. When a formulation comprises a mixture of the present resilient granules and non-resilient granules, the non-resilient granules may also comprise an active agent. Illustrative, non-limiting, examples include analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

Other illustrative, non-limiting, examples of active agents include antitussives, decongestants, alkaloids, laxatives, antacids, ion exchange resins, anti-cholesterolemics, antipyretics, analgesics including acetaminophen, aspirin, non-steroidal anti-inflammatory drugs (NSAID) and opioids, appetite suppressants, expectorants, anti-anxiety agents, anti-ulcer agents, coronary dilators, cerebral dilators, peripheral vasodilators, anti-infectives, psycho-tropics, antimanics, stimulants, gastrointestinal agents, sedatives, anti-diarrheal preparations, anti-anginal drugs, vasodilators, vasoconstrictors, migraine treatments, tranquilizers, anti-psychotics, antitumor drugs, antithrombotic drugs, hypnotics, anti-emetics, anti-nausants, anti-convulsants, neuromuscular drugs, hyper- and hypoglycemic spasmodics, uterine relaxants, antiobesity drugs, anabolic drugs, erythropoetic drugs, antiasthmatics, mucolytics, anti-uricemic drugs, and mixtures thereof. In a preferred embodiment, the active agent may be selected from the group consisting of analgesics, antibiotics, lipid regulating agent, antihistamines, antineoplastic agents, and antiviral agents.

The resilient self-adhering granules of the present invention, or non-resilient granules when present, may further comprise a nutritional active material and include, without limitation, calcium-containing materials such as calcium carbonate, stannol esters, hydroxycitric acid, vitamins, minerals, herbals, spices, and mixtures thereof. Examples of vitamins that are available as active ingredients include, without limitation, vitamin A (retinol), vitamin D (cholecalciferol), vitamin E group, vitamin K group (phylloquinones and menaquinones), thiamine, riboflavin, niacin, folic acid, cobalamins, biotin, vitamin C (ascorbic acid), and mixtures thereof. The amount of vitamin or vitamins present in the final product of the present inventive subject matter is dependent on the particular vitamin and is generally the United States' Department of Agriculture Recommended Daily Allowances (USRDA) for that vitamin. For example, if vitamin C is the active ingredient, the amount of vitamin C in the encapsulated product would be 60 milligrams, which is the USRDA of vitamin C for adults. In a preferred embodiment, the nutritional material may be zinc or calcium.

Pharmaceutical compositions according to the invention may also comprise one or more binding agents, filling agents, lubricating agents, suspending agents, emulsifiers, sweeteners, flavoring agents, preservatives, buffers, wetting agents, disintegrants, effervescent agents, and other excipients. Such excipients are well known in the art.

Resilient, self-adhering granules of the invention can be prepared by a variety of granulation techniques known in the art. For example, granulation can be accomplished by granulating in a fluidized bed and admixture comprising an active agent and one or more pharmaceutically acceptable water-soluble or water-dispersible excipients, to form a granule. Alternatively, granulation may be performed by using high-shear granulation.

The resilient self-adhering granules of the invention can be formulated into several known oral dosage forms. For example, tablets can be prepared by pharmaceutical compression or molding techniques known in the art. In addition, powders for administration can be prepared from the granules of the present invention or directly as granulated powders by any method known in the art. For example, such methods include, but are not limited to, milling, fluid bed granulation, dry granulation, direct compression, spheronization, spray congealing, and spray drying. Detailed descriptions of tabletting methods are provided in Remington: The Science and Practice of Pharmacy, 19th ed Vol. 11 (1995) (Mack Publishing Co., Pennsylvania); and Remington's Pharmaceutical Sciences, Chapter 89, pp. 1633-1658 (Mach Publishing Company, 1990), both disclosures of which are incorporated by reference herein.

In one embodiment, an active agent, and at least one pharmaceutically acceptable water-soluble or water-dispersible excipient, and, optionally, other excipients are mixed to form a blend, which is then directly compressed into tablets. For example, an active agent can be blended with tablet excipients using a V-blender or high-shear mixer to produce free-flowing compressible granules, which may be sieved for size uniformity. This may be followed by compression of the powder-granules using, for example, an automated press, or a single station press, or a high-speed tablet press.

The tablets may be further coated or uncoated. If coated, they may be sugarcoated or film-coated (to cover objectionable tastes or odors and to protect against oxidation) or coated with a release-modifying coating material such as an enteric coating. In accordance with coatings of art-known non-resilient granules tablets and caplets, a variety of coating materials and techniques are available to coat the tablets and caplets and granules of the present invention and to effect a variety of release patterns.

The present invention also provides methods to produce resilient, self-adhering granules. In one embodiment, the method may comprise of the following steps:

a) heating mixed fats and emulsifiers to about 120° F. to about 220° F. to obtain liquid consistency;

b) adding carbohydrates to the liquid of step a), optionally heating to about 120° F. to about 180° F.;

c) adding active agent, and optionally excipients selected from the group consisting of sweeteners, swelling agents, flavoring agents, binders, disintegrants, bulking agents, and mixtures thereof;

d) adding a binding agent and optionally blending;

e) lowering temperature of the composition formed in step d) to a temperature below room temperature; and

f) subjecting the composition of step e) to size-reduction methods selected from the group consisting of: milling, shearing, sieving, or a combination thereof to obtain substantially uniform sized compressible resilient granules.

In some embodiments, the method further comprises adding lubricants and other tabletting ingredients such as glidants and optionally blending and compressing into an oral dosage form.

The present invention also provides methods of administering an active agent to a subject including providing the active agent in an oral dosage form, the oral dosage form can comprise resilient self-adhering granules as previously discussed, administering the oral dosage form to the subject's oral cavity, such that the majority of the active agent can be released in the gastrointestinal tract at a point after the mouth.

In one embodiment, the tablet can be administered by breaking the tablet into two portions approximately defined by a scoring, where the breaking results in substantially no material loss. Additionally, the tablet can be reformed with substantially no material loss. In one embodiment, the oral dosage form can be characterized as not a gum or chew formulation.

The compositions and methods of the present invention can be prepared according to the examples set out below. The examples are presented for purposes of demonstrating, but not limiting, the preparation of the compounds and compositions of this invention.

### EXAMPLES

### Example 1

### Zinc Acetate and Zinc Gluconate Comprising Resilient Granules

In this Example, zinc acetate and zinc gluconate comprising resilient granules were prepared. The combined amount of elemental zinc was approximately 10.5 mg in the final compressed product. The composition had the following ingredients: mono- and di-glyceride emulsifiers (Durem 117) (60 mg); Panalite 90 DK (maltitol syrup) (30 mg); polyethylene glycol 3350 (40 mg); partially hydrogenated soy bean oil and cotton seed oil (Kaomel) (10 mg); acetylated mono- and di-glycerides (Myvacet) (50 mg); maltodextrin (Maltrin M-180) (171 mg); maltitol syrup (Lycasin HDS) (200 mg); methylcellulose (Methocel K100) (5 mg); granulated sugar (285 mg); sweeteners and colorants and flavoring aids (about 35 mg).

The fats and emulsifying agents were mixed together in a mixer and heated till the ingredients were melted. Maltitol syrup was then added to the molten fat and was mixed thoroughly. To this was added the active ingredient and mixed further for about 10 minutes. The molten and mixed mass was then extruded in an extruder and the ropes were collected into trays. The ropes then were allowed to condition at room temperature for about 6-8 hrs. The dried roped material was then milled to desired particle size. During milling, dry ice was used as needed to avoid sticking of the material. The material was then cooled in a freezer or was used directly to lubrication and compression.

The resilient material was sifted through an appropriate mesh for the desired particle size. The sifted material was then lubricated in a mixer by using a lubricant or lubricant mixture. In this particular example, the lubricant was a mixture of magnesium stearate and talc comprising about 2.5% w/w of the final composition; however, other lubricants may be used. If needed, depending on the particular active ingredient, colloidal silicon dioxide may also be used in addition to the above mixture of lubricants. If used, colloidal silicon dioxide may comprise about 3% w/w of the final composition. Sweeteners, flavors, and colorants as desired my then be added to the blending mixture. If used, sweeteners, flavors, and colorants may add up to about 5% w/w of the final composition. The mixer was run at 40 rpm and the mixing was continued for 10 minutes. The material was then frozen in a refrigerator for about 6 hrs prior to compression. The resilient granules were then compressed into tablets. The uncoated tablets comprising resilient granules were then coated in a coating pan using a coating mixture comprising Crystalac C and talc. The coating composition amounted to about 5% w/w of the final composition.

### Example 2

### Zinc Acetate Comprising Compressible Resilient Granules

Example 1 was followed to make compositions comprising zinc acetate as the active agent. The composition comprises zinc acetate: 5.6% w/w.

### Example 3

### Sumatriptan Comprising Compressible Resilient Granules

The composition comprised 20.1% w/w of sumatriptan, 24.5% w/w of mixed fats and emulsifiers, 17% w/w of maltodextrin, 23% w/w of maltitol syrup, with the remaining to include flavors, sweeteners, swelling agents, coloring agents, and lubricating agents. The sumatriptan employed is an encapsulated taste-masked formulation prepared by art-known processes such as resin-complexation, complex coacervation, polymer coating, and wax-fat coating. The sumatriptan formulation provides 100 mg per dosage form.

The process for granule preparation was as follows. Mixed fats and emulsifiers were heated to 180° F.-190° F. to obtain liquid consistency. Maltitol syrup was added to this liquid mixture and heating continued to about 150 F. The mixture was then transferred to a Sigma mixer using front blade at 60 RPM for about 1-3 minutes. The taste-masked sumatriptan, sweeteners, and swelling agents were added to this mixture, and blending continued for a few more minutes at 60 RPM. Maltodextrin and flavors were added to this mixture, and blending continued for further few minutes. Alternatively, sumatriptan may be added at this stage. The mixture was then cooled.

This was followed by size reduction using multi-mill with knife blade forward and screened to obtain uniform granule size using a size 12 screen. Lubricants and other tabletting ingredients including a glidant were added to these granules and the mixture was blended for a few minutes. The mixture was then transferred to a tabletting machine for compression into tablets of desired shape and size.

### Example 4

### Sumatriptan Comprising Compressible Resilient Granules

The process described in Example 3 was followed with the exception that sumatriptan was added at the lubrication stage.

### Example 5

### Uncoated Active Ingredient Comprising Compressible Resilient Granules

The Example of either 1 or 2 was followed except that the active agent is uncoated drug and the drug is loratadine. The loratadine is present from about 5 mg to 20 mg. The remaining ingredients of the composition are adjusted accordingly to take into consideration the total weight percentages to substantially remain the same as previously described.

### Example 6

### Compressible Resilient Granules Mixed with Coated Modified Release Granules

The Example of either 1 or 2 was followed except that the resilient compressible granules are made with no active agent. Once the compressible resilient granules are made, these are mixed with granules or powder comprising active ingredients that are coated with a delayed release coating. Olanzapine composition that is coated with a delayed release coating is used as an example to deliver 20 mg olanzapine per dosage form. Preparation of such delayed release coated olanzapine is known in the art. Granules of delayed release coated olanzapine are then mixed with compressible resilient granules at the lubrication stage into the final blend and the mixture is then compressed into a tablet as described above. The dosage form delivers modified release olanzapine.

### Example 7

### Compressible Resilient Granules Mixed with Coated Modified Release Granules

The Example of 6 was followed except that the resilient compressible granules are made with olanzapine that is not coated. These compressible granules are then mixed with granules of delayed release coated olanzapine at the lubrication stage into the final blend and the mixture is then compressed into a tablet as described above. The dosage form delivers an initial immediate release dose of olanzapine followed by delayed release olanzapine to produce therapeutic activity over a longer duration, preferably for 6-8 hrs or even longer, if desired.

### Example 8

### Compressible Resilient Granules Mixed with Coated Modified Release Granules of Two Different Actives

The Example of either 1 or 2 was followed except that the resilient compressible granules are made with the active agent comprising amlodipine. Amlodipine besylate can be used as an example to deliver 5 mg. Amlodipine may be encapsulated for taste-masking purposes if needed. Separately, compressible resilient granules comprising atorvastatin calcium are made. The atorvastatin granules are made to deliver 10, 20, 40, or 80 mg per dosage form and optionally are coated for taste-masking purposes as needed. These two compressible resilient granules are then mixed, at the lubrication stage into the final blend and the mixture is then compressed into a tablet as described above. The dosage form delivers combination of both amlodipine and atorvastatin.

### Example 9

### Compressible Resilient Granules Mixed with Coated Modified Release Granules of Two Different Actives and Different Release Rates

The Example of 8 was followed except that either amlodipine or atorvastatin may be prepared as delayed-release granules while the other drug is prepared as compressible resilient granules. The two types of granules are then mixed, at the lubrication stage into the final blend and the mixture is then compressed into a tablet as described above. The dosage form delivers an immediate release of either amlodipine or atorvastatin while providing modified release delivery of the other active.

### Example 10

### Aspirin Chew Melt

The compositions in the following Examples were prepared in accordance with the following flow chart.

Resilient granules process and flow chart.

| STAGE | OPERATION | PROCESS | EQUIPMENT |
|---|---|---|---|
| PROC ESS A: Step 1: Step 2: | Weighing/ Dispensing Melting | 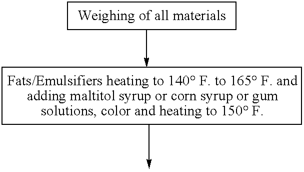 | 1. Weighing balance 2. scoops. 1. steam jacketed melter or electric heater |
| Step 3: | Mixing | 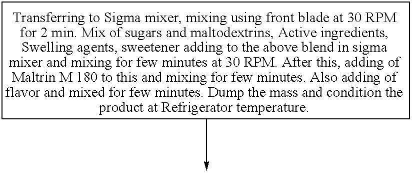 | 1. Sigma blade mixer or high shear mixer or extrusion-mixer |
| Step 4: PART B: LUBRI- CATION Step 6: | A. Conditioning and B. Size reduction c. Conditioning Lubrication and blending Condition the final blend at refrigerator temperature | | |
| # PRODUCT FORMING AND Compression | 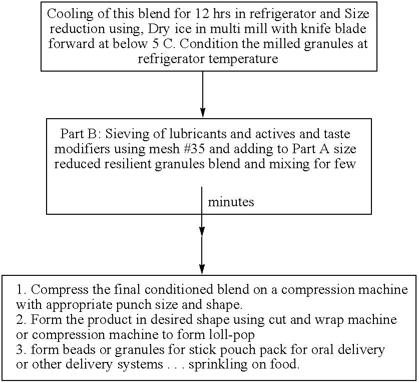 | Conditioning: Cool room or refrigerator 5 C 1. Multi mill 2. Screen #12 1. Hobart mixer 2. Screen #35 3. Conditioning 1. Tabletting machine 2. Forming | |

Objective: Sugared and sugar free version. Placebo resilient granules were made and the active was added in part B lubrication process step and the drug was not resilient granules.

### Part A

### Resilient Granules Composition

### Placebo

| | | Example | | |
|---|---|---|---|---|
| | | 1.A.1 | Example | Example |
| | | Percentage | 1.A.2 | 1.A.3 |
| | Materials by | Sugared | Percentage | Percentage |
| S. N | functionality | product | Sugar free | Sugar |
| 1. | Diluents/fillers/dry | | | |
| | binding agents | | | |
| | Maltodextrin/corn | 12.372 | — | 10.000 |
| | syrup solids | | | |
| | Saccharide/Sugar | 23.122 | — | 14.022 |
| | Polyols/Maltitol | — | 30.30 | |
| 2. | Binding/Salivating/ | | | |
| | Lubricating agents - | | | |
| | emulsifiers/fats/waxes | | | |
| | Mono and | 4.558 | 8.00 | 6.00 |
| | diglycerides | | | |
| | Polyethylene glycol | 2.930 | — | — |
| | 3350 | | | |
| | Hydrogenated soy | 2.605 | — | — |
| | bean/cottonseed oil | | | |
| | Acetylated | 5.860 | 3.00 | 4.00 |
| | monoglyceride | | | |
| | Distilled | — | 7.00 | 7.00 |
| | monoglycerides | | | |
| 3. | Taste Modifiers-1 | | | |
| | Flavor-Mint,/Fruit/ | 1.50 | 2.00 | 2.00 |
| | berry/chocolate | | | |
| | Powder | | | |
| | Potent sweetener- | 0.25 | 0.25 | 0.25 |
| | sucralose/aspartame- | | | |
| | acesulfame k | | | |
| | Souring agent-citric | — | — | — |
| | acid/ | | | |
| 4. | Liquid binding | | | |
| | systems | | | |
| | Maltitol syrup | 14.325 | 16.000 | — |
| | Corn syrup 44 be | — | — | 16.00 |
| 5. | Coloring agent | 0.078 | 0.05 | 0.078 |
| | Total | 67.600 | 66.60 | 59.35 |

Part A Process: as per process sheet prepare granules

### Part B

### Resilient Granules Composition

### Dry Blending System

| | | Example | Example | Example |
|---|---|---|---|---|
| | | 1.B.1. | 1.B.2 | 1.B.3 |
| S. N | Materials by functionality | Percentage | Percentage | percentage |
| 1. | Active Ingredients | | | |
| | Encapsulated Aspirin | 27.70 | 27.70 | 27.70 |
| | 72.2% | | | |
| 2. | Diluents/fillers/dry | | | |
| | binding agents | | | |
| | Maltodextrin/corn syrup | — | — | 1.00 |
| | solids | | | |
| | Dextrose/sugar/polyol | — | — | 5.00 |
| 3. | Lubricating agents | | | |
| | Magnesium stearate | 1.00 | 1.50 | 1.00 |
| | Talc | 0.500 | 0.50 | 0.25 |
| | Colloidal silicon dioxide | 2.00 | 2.50 | 3.00 |
| | Hydrogenated Fat/ | — | — | 1.50 |
| | emulsifier/wax | | | |
| 4. | Taste Modifiers-1 | | | |
| | Flavor- | 1.00 | 1.00 | 1.00 |
| | mint/fruit/berry/chocolate | | | |
| | Sweetener- | 0.10 | 0.10 | 0.10 |
| | sucralose/aspartame- | | | |
| | acesulfame k | | | |
| | Souring agent-citric acid | — | — | — |
| 5. | Coloring agent | 0.1 | 0.1 | 0.1 |
| | Total | 32.40 | 33.40 | 40.65 |

Part B process: Blend part A granules with part B blend in a blender or Hobart mixer. The total blend was ready for compression

Part C: Final product forming/compression:

The final blend of granules was compressed on a compression machine using oval or round shaped punches.

### Compression Tests

| S. N | Tests | Results | |
|---|---|---|---|
| 1. | Granules Flow | Very good | |
| | observation in | | |
| | machine | | |
| 2. | Weight | 2.50 grams | No weight |
| | | | variation was |
| | | | observed |
| 3. | Tablet hardness | 5 to 7 kilo | |
| | | ponds | |
| 4. | Friability | Less than | |
| | | 0.3% | |
| 5. | Tablet breakage- | No | |
| | reconnection | powder. | |
| | | Easily | |
| | | stuck back | |
| | | as original | |
| | | tablet | |

Part D: Coating. This step is optional. The product was film color coated with polymer solvent color.

### Example 11

### Pain Medication

### Hydrocodone and Ibuprofen

Objective: Sugared chewable tablet product.

### Part A

### Resilient Granules Composition

| | | Example | |
|---|---|---|---|
| | | 1.A.1 | |
| | | Percentage | Example |
| | | Sugared | 1.A.2 |
| | Materials by | product | Percentage |
| S. N | functionality | with drug | Sugar |
| 1. | Active ingredient | | |
| | Hydrocodone | 1.00 | — |
| | Ibuprofen . . . powder or | 20.00 | — |
| | granulated | | |
| 1. | Diluents/fillers/dry | | |
| | binding agents | | |
| | Maltodextrin/corn | 15.00 | 15.00 |
| | syrup solids | | |
| | Saccharide/Sugar | 18.45 | 14.35 |
| | Polyols/Maltitol/ | — | — |
| | sorbitol/isomalt | | |
| 2. | Binding/Salivating/ | | |
| | Lubricating agents - | | |
| | waxes/fats/waxes | | |
| | Mono and | 8.00 | 8.00 |
| | diglycerides | | |
| | Polyethylene glycol | 4.00 | — |
| | 8000 | | |
| | Hydrogenated soy | 3.00 | — |
| | bean/cottonseed oil | | |
| | Acetylated | 4.00 | 3.00 |
| | monoglyceride | | |
| | Distilled | — | 7.00 |
| | monoglycerides | | |
| 3. | Taste Modifiers-1 | | |
| | Flavor-Mint,/Fruit/ | 1.50 | 2.00 |
| | berry/chocolate | | |
| | Powder | | |
| | Potent sweetener- | 0.25 | 0.25 |
| | sucralose/aspartame- | | |
| | acesulfame k | | |
| | Souring agent-citric | — | — |
| | acid/ | | |
| 4. | Liquid binding | | |
| | systems | | |
| | Maltitol syrup | 19.50 | 17.00 |
| | Corn syrup 44 be | — | — |
| | Polysaccharide/ | 0.50 | 0.50 |
| | Gums | | |
| 5. | Coloring agent | 0.10 | 0.10 |
| | Total | 95.30 | 60.00 |

Part A Process: Prepare granules as per the process sheet.

### Part B

### Resilient Granules Composition Dry Blend With and Without Actives System

| | | Example | |
|---|---|---|---|
| | | 1.B.1. | Example 1.B.2 |
| S. N | Materials by functionality | Percentage | Percentage |
| 1. | Active Ingredients | | |
| | Hydrocodone | — | 0.66 |
| | Ibuprofen powder or | — | 20.00 |
| | granulated | | |
| 2. | Diluents/fillers/dry | | |
| | binding agents | | |
| | Maltodextrin/corn syrup | — | — |
| | solids | | |
| | Dextrose/sugar/polyol | — | 13.36 |
| 3. | Lubricating agents | | |
| | Magnesium stearate | 1.00 | 1.50 |
| | Talc | 0.500 | 0.50 |
| | Silicon dioxide | 2.00 | 2.50 |
| | Hydrogenated Fat/ | — | — |
| | emulsifier/wax | | |
| 4. | Taste Modifiers-1 | | |
| | Flavor- | 1.00 | 1.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.1 | 0.1 |
| | Total | 4.70 | 40.00 |

Part B process: Blend part A granules with part B blend in a blender or Hobart mixer. The total blend was ready for compression.

Part C: Final product forming

1. Compression:

The final blend of granules was compressed on a compression machine using round beveled edge punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product was made on specially designed cut/wrap machine for inserting stick for lollipop.

Part D: Coating. This step is optional. The product was film color coated with polymer solvent color. Product was also coated with sugar coating.

### Example 12

### Pain Medication

### Fentanyl Citrate

Sugared and sugar free version in a lollipop form or chewable formed product.

### Part A

### Resilient Granules Composition

| | | Example | | |
|---|---|---|---|---|
| | | 1.A.1 | Example | Example |
| | | Percentage | 1.A.2 | 1.A.3 |
| | | Sugared | Percentage | Percentage |
| | Materials by | product | Sugar free | Sugar- |
| S. N | functionality | with drug | with drug | placebo |
| 1. | Active ingredient | | | |
| | Fentanyl citrate- | 0.04 | 0.04 | — |
| | control substance | | | |
| 1. | Diluents/fillers/dry | | | |
| | binding agents | | | |
| | Maltodextrin/corn | 20.00 | — | 20.00 |
| | syrup solids | | | |
| | Saccharide/Sugar | 34.16 | — | 29.16 |
| | Polyols/Maltitol/ | — | 52.41 | — |
| | sorbitol/isomalt | | | |
| 2. | Binding/Salivating/ | | | |
| | Lubricating agents - | | | |
| | waxes/fats/waxes | | | |
| | Mono and | 8.00 | 8.00 | 6.00 |
| | diglycerides | | | |
| | Polyethylene glycol | 4.00 | — | — |
| | 8000 | | | |
| | Hydrogenated soy | 3.00 | — | — |
| | bean/cottonseed oil | | | |
| | Acetylated | 4.00 | 3.00 | 4.00 |
| | monoglyceride | | | |
| | Distilled | — | 7.00 | 7.00 |
| | monoglycerides | | | |
| 3. | Taste Modifiers-1 | | | |
| | Flavor-Mint,/Fruit/ | 1.50 | 2.00 | 2.00 |
| | berry/chocolate | | | |
| | Powder | | | |
| | Potent sweetener- | 0.25 | 0.25 | 0.25 |
| | sucralose,/ | | | |
| | aspartame- | | | |
| | acesulfame k | | | |
| | Souring agent-citric | — | — | — |
| | acid/ | | | |
| 4. | Liquid binding | | | |
| | systems | | | |
| | Maltitol syrup | 19.50 | 21.000 | 18.00 |
| | Corn syrup 44 be | — | — | — |
| | Polysaccharide/ | 0.50 | 0.50 | 0.50 |
| | Gums | | | |
| 5. | Coloring agent | 0.10 | 0.10 | 0.10 |
| | Total | 95.30 | 94.30 | 87.01 |

Part A Process: Prepare granules as per the process sheet.

### Part B

### Resilient Granules Composition

### Dry Blending System

| | | | | Example |
|---|---|---|---|---|
| | | Example | Example | 1.B.3 |
| | | 1.B.1. | 1.B.2 | Percentage |
| S. N | Materials by functionality | Percentage | Percentage | With drug |
| 1. | Active Ingredients | | | |
| | Fentanyl citrate | — | — | 0.04 |
| 2. | Diluents/fillers/dry | | | |
| | binding agents | | | |
| | Maltodextrin/corn syrup | — | — | 1.00 |
| | solids | | | |
| | Dextrose/sugar/polyol | — | — | 5.00 |
| 3. | Lubricating agents | | | |
| | Magnesium stearate | 1.00 | 1.50 | 1.00 |
| | Talc | 0.500 | 0.50 | 0.25 |
| | Colloidal silicon dioxide | 2.00 | 2.50 | 3.00 |
| | Hydrogenated Fat/ | — | — | 1.50 |
| | emulsifier/wax | | | |
| 4. | Taste Modifiers-1 | | | |
| | Flavor- | 1.00 | 1.00 | 1.00 |
| | mint/fruit/berry/chocolate | | | |
| | Sweetener- | 0.10 | 0.10 | 0.10 |
| | sucralose/aspartame- | | | |
| | acesulfame k | | | |
| | Souring agent-citric acid | — | — | — |
| 5. | Coloring agent | 0.1 | 0.1 | 0.1 |
| | Total | 4.70 | 5.70 | 12.99 |

Part B process: Blend part A granules with part B blend in a blender or Hobart mixer. The total blend was ready for compression Part C: Final product forming

1. Compression:

The final blend of granules was compressed on a compression machine using special shaped cone shaped tablet with hole in the middle shaped punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product was made on specially designed cut/wrap machine for inserting stick for lollipop.

Part D: Coating. This step is optional. The product was film color coated with polymer solvent color. Product was also coated with sugar coating.

### Example 13

### Throat Antiseptic

### Benzocaine and Menthol Chew Tablet

Objective: Sugared and sugar free version with variable.

### Part A

### Resilient Granules Composition

| | | Example 1 | Example |
|---|---|---|---|
| | | Percentage | 1.2 |
| | Materials by | Sugared | Percentage |
| S. N | functionality | product | Sugar free |
| 1. | Active Ingredients | | |
| | Benzocaine | 0.600 | — |
| | Menthol as | 2.500 | — |
| | encapsulated powder | | |
| 2. | Diluents/fillers/dry | | |
| | binding agents | | |
| | Maltodextrin/corn | 17.090 | — |
| | syrup solids | | |
| | Saccharide/Sugar | 34.290 | — |
| | Polyols/Maltitol | — | 45.38 |
| 3. | Binding/Salivating/ | | |
| | Lubricating agents - | | |
| | waxes/fats/waxes | | |
| | Mono and | 6.00 | 8.00 |
| | diglycerides | | |
| | Polyethylene glycol | 4.00 | 4.00 |
| | 3350 | | |
| | Hydrogenated soy | 2.00 | — |
| | bean/cottonseed oil | | |
| | Acetylated | 4.00 | — |
| | monoglyceride | | |
| | Distilled | 3.00 | 7.00 |
| | monoglycerides | | |
| 4. | Taste Modifiers-1 | | |
| | Flavor-Mint,/Fruit/ | 1.50 | 1.50 |
| | berry/chocolate | | |
| | Powder | | |
| | Potent sweetener- | 0.25 | 0.25 |
| | sucralose,/ | | |
| | aspartame- | | |
| | acesulfame k | | |
| | Souring agent-citric | — | — |
| | acid/ | | |
| 5. | Liquid binding | | |
| | systems | | |
| | Maltitol syrup | — | 19.00 |
| | Corn syrup 44 be | 18.00 | — |
| | Polysaccharide/gums | 0.50 | 0.50 |
| 6. | Coloring agent | 0.02 | 0.02 |
| | Total | 93.75 | 85.65 |

Part A Process: prepare granules as per the process sheet

### Part B

### Resilient Granules Composition

### Dry Blending System

| | | | Example |
|---|---|---|---|
| | | | B.2 |
| | | | Percentage |
| | | Example | Actives |
| | | B.1. | added in |
| S. N | Materials by functionality | Percentage | lubrication |
| 1. | Active Ingredients | — | |
| | Benzocaine | — | 0.60 |
| | Menthol as | — | 2.50 |
| | encapsulated powder | | |
| 2. | Diluents/fillers/dry | | |
| | binding agents | | |
| | Maltodextrin/corn syrup | — | — |
| | solids | | |
| | Dextrose/sugar/polyol | — | 5.00 |
| 3. | Lubricating agents | | |
| | Magnesium stearate | 1.00 | 1.50 |
| | Talc | 1.00 | 0.50 |
| | Colloidal silicon dioxide | 3.00 | 2.50 |
| | Hydrogenated Fat/ | — | — |
| | emulsifier/wax | | |
| 4. | Taste Modifiers-1 | | |
| | Flavor - | 3.00 | 3.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.15 | 0.15 |
| | Total | 6.25 | 14.35 |

Part B process: Blend actives, lubricants, taste modifiers and color in a Hobart mixer Mix Part A resilient granules and part B lubricating blend in a Hobart mixer.

Part C: Final product forming/compression:

Compress the blend with good hardness using rectangular or round or concave punch. The results are given below

Part C: Final product forming/compression:

The final blend of granules was compressed on a compression machine using oval or round shaped punches.

### Compression Tests

| S. N | Tests | Results | |
|---|---|---|---|
| 1. | Granules Flow | Very good | |
| | observation in | | |
| | machine | | |
| 2. | Weight | 1.00 grams | No weight |
| | | | variation was |
| | | | observed |
| 3. | Tablet hardness | 3 to 5 kilo | Consistent |
| | | ponds | hardness |
| 4. | Friability | Less than | No powder was |
| | | 0.2% | observed |
| 5. | Tablet breakage- | No | |
| | reconnection. | powder. | |
| | Tablet was broken | Easily | |
| | in the middle with | stuck back | |
| | fingers into two | as original | |
| | pieces and was | tablet | |
| | reattached to | | |
| | original shape | | |

Part D: Coating. This step is optional. The product was film color coated with polymer solvent color and talc is added. Product was also coated with aqueous sugar coating

Part D: Coating

### Example 14

### Product

### Antacid and Calcium Supplement

Objective: Sugared and sugar free version with variable

### Part A

### Resilient Granules Composition

| | | Example 1 | Example |
|---|---|---|---|
| | | Percentage | 1.2 |
| | Materials by | Sugared | Percentage |
| S. N | functionality | product | Sugar free |
| 1. | Active Ingredients | | |
| | Calcium carbonate | 40.80 | 40.80 |
| 2. | Diluents/fillers/dry | | |
| | binding agents | | |
| | Maltodextrin/corn | 6.05 | — |
| | syrup solids | | |
| | Saccharide/Sugar | — | — |
| | Polyols/Maltitol | — | 6.55 |
| 3. | Binding/Salivating/ | | |
| | Lubricating agents - | | |
| | waxes/fats/waxes | | |
| | Mono and | 6.00 | 8.00 |
| | diglycerides | | |
| | Polyethylene glycol | 4.00 | — |
| | 3350 | | |
| | Hydrogenated soy | 4.00 | — |
| | bean/cottonseed oil | | |
| | Acetylated | 7.00 | 5.00 |
| | monoglyceride | | |
| | Distilled | — | 7.00 |
| | monoglycerides | | |
| 4. | Taste Modifiers-1 | | |
| | Flavor - Mint,/Fruit/ | 2.00 | 2.00 |
| | berry/chocolate | | |
| | Powder | | |
| | Potent sweetener- | 0.25 | 0.25 |
| | sucralose,/ | | |
| | aspartame- | | |
| | acesulfame k | | |
| | Dry non fat milk solids | 5.00 | 5.00 |
| 5. | Liquid binding | | |
| | systems | | |
| | Maltitol syrup | — | 19.00 |
| | Corn syrup 44 be | 19.00 | — |
| | Polysaccharide/gums | 0.5 | 0.50 |
| | Glycerin | — | — |
| 6. | Coloring agent | 0.05 | 0.05 |
| | Total | 93.75 | 94.25 |

Part A Process: Prepare granules as per the process sheet

### Part B

### Resilient Granules Composition

### Dry Lubricating Blending System

| | | Example | Example |
|---|---|---|---|
| | | B.1. | B.2 |
| S. N | Materials by functionality | Percentage | Percentage |
| 1. | Active Ingredients | | |
| 2. | Diluents/fillers/dry | | |
| | binding agents | | |
| | Maltodextrin/corn syrup | 3.00 | — |
| | solids | | |
| | Dextrose/sugar/polyol | — | — |
| 3. | Lubricating agents | | |
| | Magnesium stearate | 1.00 | 1.50 |
| | Talc | — | 0.50 |
| | Colloidal silicon dioxide | 1.00 | 2.50 |
| | Hydrogenated Fat/ | — | — |
| | emulsifier/wax | | |
| 4. | Taste Modifiers-1 | | |
| | Flavor - | 1.00 | 1.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.15 | 0.15 |
| | Total | 6.25 | 5.75 |

Part B process: Dry blend ingredients in a Hobart mixer

Blend part a resilient granules and part B mix in Hobart mixer.

Part C: Final product forming/compression:

Part C: Final product forming/compression:

The final blend of granules was compressed on a compression machine using oval or round shaped punches.

### Compression Tests

| S. N | Tests | Results | |
|---|---|---|---|
| 1. | Granules Flow | Very good | |
| | observation in | | |
| | machine | | |
| 2. | Weight | 2.50 grams | No weight |
| | | | variation was |
| | | | observed |
| 3. | Tablet hardness | 5 to 7 kilo | |
| | | ponds | |
| 4. | Friability | Less than | |
| | | 0.4% | |
| 5. | Tablet breakage- | No | |
| | reconnection. | powder. | |
| | Break the tablet | Easily | |
| | with fingers into two | stuck back | |
| | parts and reconnect | as original | |
| | them by fingers | tablet | |
| | pressure into | | |
| | original shape | | |

Part D: Coating. This step is optional. The product was film color coated with polymer solvent color or sugar or sugar free coated

### Example 15

### Product

### Pain Medication

### Hydrocodone and Acetaminophen

Objective: Sugared chewable tablet product.

### Part A

### Resilient Granules Composition

| | | Example | |
|---|---|---|---|
| | | 1.A.1 | |
| | | % Sugared | Example |
| | Materials by | product | 1.A.2 |
| S. N | functionality | with drug | % Sugar |
| 1. | Active ingredient | | |
| | Hydrocodone | 0.66 | — |
| | Acetaminophen | 21.66 | — |
| 1. | Diluents/fillers/dry | | |
| | binding agents | | |
| | Maltodextrin/corn | 15.00 | 15.00 |
| | syrup solids | | |
| | Saccharide/Sugar | 17.20 | 7.15 |
| | Polyols/Maltitol/ | — | |
| | sorbitol/isomalt | | |
| 2. | Binding/Salivating/ | | |
| | Lubricating agents - | | |
| | waxes/fats/waxes | | |
| | Mono and | 8.00 | 8.00 |
| | diglycerides | | |
| | Polyethylene glycol | 4.00 | — |
| | 8000 | | |
| | Hydrogenated soy | 3.00 | — |
| | bean/cottonseed oil | | |
| | Acetylated | 4.00 | 3.00 |
| | monoglyceride | | |
| | Distilled | — | 7.00 |
| | monoglycerides | | |
| 3. | Taste Modifiers-1 | | |
| | Flavor - Mint,/Fruit/ | 1.50 | 2.00 |
| | berry/chocolate | | |
| | Powder | | |
| | Potent sweetener- | 0.25 | 0.25 |
| | sucralose,/ | | |
| | aspartame- | | |
| | acesulfame k | | |
| | Souring agent-citric | — | — |
| | acid/ | | |
| 4. | Liquid binding | | |
| | systems | | |
| | Maltitol syrup | 19.50 | 17.00 |
| | Corn syrup 44 be | — | — |
| | Polysaccharide/ | 0.50 | 0.50 |
| | Gums | | |
| 5. | Coloring agent | 0.10 | 0.10 |
| | Total | 95.30 | 60.00 |

Part A Process: Prepare granules as per the process sheet.

### Part B

### Resilient Granules Composition Dry Blend with and without Actives System

| | | Example | |
|---|---|---|---|
| | | 1.B.1. | Example 1.B.2 |
| S. N | Materials by functionality | Percentage | Percentage |
| 1. | Active Ingredients | | |
| | Hydrocodone | — | 0.66 |
| | Acetaminophen | — | 21.66 |
| 2. | Diluents/fillers/dry | | |
| | binding agents | | |
| | Maltodextrin/corn syrup | — | — |
| | solids | | |
| | Dextrose/sugar/polyol | — | 11.98 |
| 3. | Lubricating agents | | |
| | Magnesium stearate | 1.00 | 1.50 |
| | Talc | 0.500 | 0.50 |
| | Silicon dioxide | 2.00 | 2.50 |
| | Hydrogenated Fat/ | — | — |
| | emulsifier/wax | | |
| 4. | Taste Modifiers-1 | | |
| | Flavor - | 1.00 | 1.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.1 | 0.1 |
| | Total | 4.70 | 40.00 |

Part B process: Blend part A granules with part B blend in a blender or Hobart mixer.

The total blend is ready for compression

Part C: Final product forming

1. Compression:

The final blend of granules was compressed on a compression machine using round beveled edge punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product is made on specially designed cut/wrap machine for inserting stick for lollipop.

Part D: Coating. This step is optional. The product was film color coated with polymer solvent color. Product was also coated with sugar coating.

### Example 16

### Pain Medication

### Oxycodone and Acetaminophen

Objective: Sugared chewable tablet product.

### Part A

### Resilient Granules Composition

| | | Example | |
|---|---|---|---|
| | | 1.A.1 | |
| | | % Sugared | Example |
| | Materials by | product | 1.A.2 |
| S. N | functionality | with drug | % Sugar |
| 1. | Active ingredient | | |
| | Oxycodone | 0.33 | — |
| | Acetaminophen | 21.66 | — |
| 1. | Diluents/fillers/dry | | |
| | binding agents | | |
| | Maltodextrin/corn | 15.00 | 15.00 |
| | syrup solids | | |
| | Saccharide/Sugar | 17.55 | 7.15 |
| | Polyols/Maltitol/ | — | |
| | sorbitol/isomalt | | |
| 2. | Binding/Salivating/ | | |
| | Lubricating agents - | | |
| | waxes/fats/waxes | | |
| | Mono and | 8.00 | 8.00 |
| | diglycerides | | |
| | Polyethylene glycol | 4.00 | — |
| | 8000 | | |
| | Hydrogenated soy | 3.00 | — |
| | bean/cottonseed oil | | |
| | Acetylated | 4.00 | 3.00 |
| | monoglyceride | | |
| | Distilled | — | 7.00 |
| | monoglycerides | | |
| 3. | Taste Modifiers-1 | | |
| | Flavor - Mint,/Fruit/ | 1.50 | 2.00 |
| | berry/chocolate | | |
| | Powder | | |
| | Potent sweetener- | 0.25 | 0.25 |
| | sucralose,/ | | |
| | aspartame- | | |
| | acesulfame k | | |
| | Souring agent-citric | — | — |
| | acid/ | | |
| 4. | Liquid binding | | |
| | systems | | |
| | Maltitol syrup | 19.50 | 17.00 |
| | Corn syrup 44 be | — | — |
| | Polysaccharide/ | 0.50 | 0.50 |
| | Gums | | |
| 5. | Coloring agent | 0.10 | 0.10 |
| | Total | 95.30 | 60.00 |

Part A Process: Prepare granules as per the process sheet.

### Part B

### Resilient Granules Composition Dry Blend with and without Actives System

| | | Example | |
|---|---|---|---|
| | | 1.B.1. | Example 1.B.2 |
| S. N | Materials by functionality | Percentage | Percentage |
| 1. | Active Ingredients | | |
| | Hydrocodone | — | 0.33 |
| | Acetaminophen | — | 21.66 |
| 2. | Diluents/fillers/dry | | |
| | binding agents | | |
| | Maltodextrin/corn syrup | — | — |
| | solids | | |
| | Dextrose/sugar/polyol | — | 12.31 |
| 3. | Lubricating agents | | |
| | Magnesium stearate | 1.00 | 1.50 |
| | Talc | 0.500 | 0.50 |
| | Silicon dioxide | 2.00 | 2.50 |
| | Hydrogenated Fat/ | — | — |
| | emulsifier/wax | | |
| 4. | Taste Modifiers-1 | | |
| | Flavor - | 1.00 | 1.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.1 | 0.1 |
| | Total | 4.70 | 40.00 |

Part B process: Blend part A granules with part B blend in a blender or Hobart mixer. The total blend was ready for compression

Part C: Final product forming

1. Compression:

The final blend of granules was compressed on a compression machine using round beveled edge punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product is made on specially designed cut/wrap machine for inserting stick for lollipop.

Part D: Coating. This step is optional. The product was film color coated with polymer solvent color. Product was also coated with sugar coating.

### Example 17

### Product

### Glucosamine and Chondroitin

Objective: Sugared and sugar free version with variable

### Part A

### Resilient Granules Composition with Actives and Encapsulated Active

| | | Example | | |
|---|---|---|---|---|
| | | 3.1 | Example | Example |
| | | Percentage | 3.2 | 3.3 |
| | Materials by | Sugared | Percentage | Percentage |
| S. N | functionality | product | Sugar free | Sugar free |
| 1. | Active Ingredients | | | |
| | Glucosamine HCl | 30.77 | 30.77 | 3077 |
| | encapsulation 65.0% | | | |
| | Chondroitin sulfate | 16.00 | 16.00 | 16.00 |
| 2. | Diluents/fillers/dry | | | |
| | binding agents | | | |
| | Maltodextrin/corn | 5.00 | — | — |
| | syrup solids | | | |
| | Saccharide/Sugar | 2.68 | — | — |
| | Polyols/Maltitol | — | 11.55 | 17.68 |
| 3. | Binding/Salivating/ | | | |
| | Lubricating agents - | | | |
| | waxes/fats/waxes | | | |
| | Mono and | 10.00 | 8.00 | 6.00 |
| | diglycerides | | | |
| | Polyethylene glycol | — | — | — |
| | 3350 | | | |
| | Hydrogenated soy | — | — | 3.00 |
| | bean/cottonseed oil | | | |
| | Acetylated | — | — | — |
| | monoglyceride | | | |
| | Distilled | 5.00 | 7.00 | 7.00 |
| | monoglycerides | | | |
| 4. | Taste Modifiers-1 | | | |
| | Flavor - Mint,/Fruit/ | 150 | 1.50 | 1.50 |
| | berry/chocolate | | | |
| | Powder | | | |
| | Potent sweetener- | 0.1 | 0.100 | 0.100 |
| | sucralose,/ | | | |
| | aspartame- | | | |
| | acesulfame k | | | |
| | Souring agent-citric | 1.25 | 1.25 | 1.25 |
| | acid/ | | | |
| 5. | Liquid binding | | | |
| | systems | | | |
| | Maltitol syrup | — | 19.00 | — |
| | Corn syrup 44 be | 15.00 | — | — |
| | Polysaccharide/gum | — | — | 7.00 |
| | solution | | | |
| | Glycerin | — | — | 1.00 |
| 6. | Coloring agent | 0.02 | 0.02 | 0.02 |
| | Total | 87.32 | 92.32 | 91.32 |

Part A Process: Prepare granules as per process sheet

### Part B

### Resilient Granules Composition

### Dry Lubricating Blend System

| | | Example | Example | Example |
|---|---|---|---|---|
| | | B.1. | B.2 | B.3 |
| S. N | Materials by functionality | Percentage | Percentage | percentage |
| 1. | Diluents/fillers/dry | | | |
| | binding agents | | | |
| | Maltodextrin/corn syrup | — | — | 1.00 |
| | solids | | | |
| | Dextrose/sugar/polyol | 5.00 | — | — |
| 2. | Lubricating agents | | | |
| | Magnesium stearate | 1.50 | 1.50 | 1.5 |
| | Talc | 1.00 | 1.00 | 1.00 |
| | Colloidal silicon dioxide | 3.00 | 3.00 | 2.00 |
| | Hydrogenated Fat/ | — | — | 1.00 |
| | emulsifier/wax | | | |
| 3. | Taste Modifiers-1 | | | |
| | Flavor- | 1.00 | 1.00 | 1.00 |
| | mint/fruit/berry/chocolate | | | |
| | Sweetener- | 0.15 | 0.15 | 0.15 |
| | sucralose/aspartame- | | | |
| | acesulfame k | | | |
| | Souring agent-citric acid | 1.00 | 1.00 | 1.00 |
| 4. | Coloring agent | 0.03 | 0.03 | 0.03 |
| | Total | 12.68 | 7.68 | 8.68 |

Part B process: Blend part a granules with part B granules in Hobart bowl mixer.

Part C: Final product forming/compression:

Blend is compressed using oval shaped or round beveled edged punches. The results are given below

Part C: Final product forming/compression:

The final blend of granules was compressed on a compression machine using oval or round shaped punches.

### Compression Tests

| S. N | Tests | Results | |
|---|---|---|---|
| 1. | Granules Flow | Very good | |
| | observation in | | |
| | machine | | |
| 2. | Weight | 2.50 grams | No weight |
| | | | variation was |
| | | | observed |
| 3. | Tablet hardness | 5 to 7 kilo | |
| | | ponds | |
| 4. | Friability | Less than | |
| | | 0.5% | |
| 5. | Tablet breakage- | No | Formed into |
| | reconnection. | powder. | original tablet |
| | Break the tablet | Easily | |
| | into two pieces with | stuck back | |
| | fingers and | as original | |
| | reconnect. | tablet | |

Part D: Coating. This step is optional. The product was film color coated with polymer solvent color

### Example 18

### Throat Antiseptic

### Benzocaine and Menthol Chew Tablet

Objective: Sugared and sugar free version with variable

### Part A

### Resilient Granules Composition

| | | Example 1 | Example |
|---|---|---|---|
| | | Percentage | 1.2 |
| | Materials by | Sugared | Percentage |
| S. N | functionality | product | Sugar free |
| 1. | Active Ingredients | | |
| | Benzocaine | 0.600 | — |
| | Menthol as | 2.500 | — |
| | encapsulated powder | | |
| 2. | Diluents/fillers/dry | | |
| | binding agents | | |
| | Maltodextrin/corn | 17.090 | — |
| | syrup solids | | |
| | Saccharide/Sugar | 34.290 | — |
| | Polyols/Maltitol | — | 45.38 |
| 3. | Binding/Salivating/ | | |
| | Lubricating agents - | | |
| | waxes/fats/waxes | | |
| | Mono and | 6.00 | 8.00 |
| | diglycerides | | |
| | Polyethylene glycol | 4.00 | 4.00 |
| | 3350 | | |
| | Hydrogenated soy | 2.00 | — |
| | bean/cottonseed oil | | |
| | Acetylated | 4.00 | — |
| | monoglyceride | | |
| | Distilled | 3.00 | 7.00 |
| | monoglycerides | | |
| 4. | Taste Modifiers-1 | | |
| | Flavor - Mint,/Fruit/ | 1.50 | 1.50 |
| | berry/chocolate | | |
| | Powder | | |
| | Potent sweetener- | 0.25 | 0.25 |
| | sucralose,/ | | |
| | aspartame- | | |
| | acesulfame k | | |
| | Souring agent-citric | — | — |
| | acid/ | | |
| 5. | Liquid binding | | |
| | systems | | |
| | Maltitol syrup | — | 19.00 |
| | Corn syrup 44 be | 18.00 | — |
| | Polysaccharide/gums | 0.50 | 0.50 |
| 6. | Coloring agent | 0.02 | 0.02 |
| | Total | 93.75 | 85.65 |

Part A Process: prepare granules as per the process sheet

### Part B

### Resilient Granules Composition

### Dry Blending System

| | | | Example |
|---|---|---|---|
| | | | B.2 |
| | | | Percentage |
| | | Example | Actives |
| | | B.1. | added in |
| S. N | Materials by functionality | Percentage | lubrication |
| 1. | Active Ingredients | — | |
| | Benzocaine | — | 0.60 |
| | Menthol as | — | 2.50 |
| | encapsulated powder | | |
| 2. | Diluents/fillers/dry | | |
| | binding agents | | |
| | Maltodextrin/corn syrup | — | — |
| | solids | | |
| | Dextrose/sugar/polyol | — | 5.00 |
| 3. | Lubricating agents | | |
| | Magnesium stearate | 1.00 | 1.50 |
| | Talc | 1.00 | 0.50 |
| | Colloidal silicon dioxide | 3.00 | 2.50 |
| | Hydrogenated Fat/ | — | — |
| | emulsifier/wax | | |
| 4. | Taste Modifiers-1 | | |
| | Flavor- | 3.00 | 3.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.15 | 0.15 |
| | Total | 6.25 | 14.35 |

Part B process: Blend actives, lubricants, taste modifiers and color in a Hobart mixer Mix Part A resilient granules and part B lubricating blend in a Hobart mixer.

Part C: Final product forming/compression:

Compress the blend with good hardness using rectangular or round or concave punch. The results are given below

Part C: Final product forming/compression:

The final blend of granules was compressed on a compression machine using oval or round shaped punches.

### Compression Tests

| S. N | Tests | Results | |
|---|---|---|---|
| 1. | Granules Flow | Very good | |
| | observation in | | |
| | machine | | |
| 2. | Weight | 1.00 grams | No weight |
| | | | variation was |
| | | | observed |
| 3. | Tablet hardness | 3 to 5 kilo | Consistent |
| | | ponds | hardness |
| 4. | Friability | Less than | No powder was |
| | | 0.2% | observed |
| 5. | Tablet breakage- | No | |
| | reconnection. | powder. | |
| | Tablet was broken | Easily | |
| | in the middle with | stuck back | |
| | fingers into two | as original | |
| | pieces and was | tablet | |
| | reattached to | | |
| | original shape | | |

Part D: Coating. This step is optional. The product was film color coated with polymer solvent color and talc is added. Product was also coated with aqueous sugar coating

Part D: Coating

### Example 19

### Pain Medication

### Fentanyl Citrate

Objective: Sugared and sugar free version in a lollipop form or chewable formed product.

### Part A

### Resilient Granules Composition

| | | Example | Example | |
|---|---|---|---|---|
| | | 1.A.1 | 1.A.2 | Example |
| | | % Sugared | % Sugar | 1.A.3 |
| | Materials by | product | free with | % Sugar- |
| S. N | functionality | with drug | drug | placebo |
| 1. | Active ingredient | | | |
| | Fentanyl citrate- | 0.04 | 0.04 | — |
| | control substance | | | |
| 1. | Diluents/fillers/dry | | | |
| | binding agents | | | |
| | Maltodextrin/corn | 20.00 | — | 20.00 |
| | syrup solids | | | |
| | Saccharide/Sugar | 34.16 | — | 29.16 |
| | Polyols/Maltitol/ | — | 52.41 | — |
| | sorbitol/isomalt | | | |
| 2. | Binding/Salivating/ | | | |
| | Lubricating agents - | | | |
| | waxes/fats/waxes | | | |
| | Mono and | 8.00 | 8.00 | 6.00 |
| | diglycerides | | | |
| | Polyethylene glycol | 4.00 | — | — |
| | 8000 | | | |
| | Hydrogenated soy | 3.00 | — | — |
| | bean/cottonseed oil | | | |
| | Acetylated | 4.00 | 3.00 | 4.00 |
| | monoglyceride | | | |
| | Distilled | — | 7.00 | 7.00 |
| | monoglycerides | | | |
| 3. | Taste Modifiers-1 | | | |
| | Flavor - Mint,/Fruit/ | 1.50 | 2.00 | 2.00 |
| | berry/chocolate | | | |
| | Powder | | | |
| | Potent sweetener- | 0.25 | 0.25 | 0.25 |
| | sucralose,/ | | | |
| | aspartame- | | | |
| | acesulfame k | | | |
| | Souring agent-citric | — | — | — |
| | acid/ | | | |
| 4. | Liquid binding | | | |
| | systems | | | |
| | Maltitol syrup | 19.50 | 21.000 | 18.00 |
| | Corn syrup 44 be | — | — | — |
| | Polysaccharide/ | 0.50 | 0.50 | 0.50 |
| | Gums | | | |
| 5. | Coloring agent | 0.10 | 0.10 | 0.10 |
| | Total | 95.30 | 94.30 | 87.01 |

Part A Process: Prepare granules as per the process sheet.

### Part B

### Resilient Granules Composition

### Dry Blending System

| | | Example | Example | Example 1.B.3 |
|---|---|---|---|---|
| | Materials by | 1.B.1. | 1.B.2 | Percentage |
| S. N | functionality | Percentage | Percentage | With drug |
| 1. | Active Ingredients | | | |
| | Fentanyl citrate | — | — | 0.04 |
| 2. | Diluents/fillers/dry | | | |
| | binding agents | | | |
| | Maltodextrin/corn syrup | — | — | 1.00 |
| | solids | | | |
| | Dextrose/sugar/polyol | — | — | 5.00 |
| 3. | Lubricating agents | | | |
| | Magnesium stearate | 1.00 | 1.50 | 1.00 |
| | Talc | 0.500 | 0.50 | 0.25 |
| | Colloidal silicon dioxide | 2.00 | 2.50 | 3.00 |
| | Hydrogenated Fat/ | — | — | 1.50 |
| | emulsifier/wax | | | |
| 4. | Taste Modifiers-1 | | | |
| | Flavor- | 1.00 | 1.00 | 1.00 |
| | mint/fruit/berry/chocolate | | | |
| | Sweetener- | 0.10 | 0.10 | 0.10 |
| | sucralose/aspartame- | | | |
| | acesulfame k | | | |
| | Souring agent-citric acid | — | — | — |
| 5. | Coloring agent | 0.1 | 0.1 | 0.1 |
| | Total | 4.70 | 5.70 | 12.99 |

Part B process: Blend part A granules with part B blend in a blender or Hobart mixer.

The total blend is ready for compression

Part C: Final product forming

1. Compression:

The final blend of granules was compressed on a compression machine using special shaped cone shaped tablet with hole in the middle shaped punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product is made on specially designed cut/wrap machine for inserting stick for lollipop.

Part D: Coating. This step is optional. The product was film color coated with polymer solvent color. Product was also coated with sugar coating.

### Example 20

### Zinc Salts Chew Tablet

Objective: Sugared product with Active added in part a resilient granules

### Part A

### Resilient Granules Composition

| | | Example 1 | Example 2 |
|---|---|---|---|
| | | Percentage | Percentage |
| | Materials by | Sugared | Sugared |
| S. N | functionality | product | product |
| 1. | Active Ingredients | | |
| | Zinc Acetate | 2.750 | 2.750 |
| | dehydrate | | |
| | Zinc Gluconate | 1.910 | — |
| 2. | Diluents/fillers/dry | | |
| | binding agents | | |
| | Maltodextrin/corn | 19.15 | 20.55 |
| | syrup solids | | |
| | Saccharide/Sugar | 25.00 | 30.00 |
| | crystals | | |
| 3. | Binding/Salivating/ | | |
| | Lubricating agents - | | |
| | waxes/fats/waxes | | |
| | Mono and | 6.00 | 8.00 |
| | diglycerides | | |
| | Polyethylene glycol | 4.00 | — |
| | 3350 | | |
| | Hydrogenated soy | 4.00 | — |
| | bean/cottonseed oil | | |
| | Acetylated | 7.00 | 5.00 |
| | monoglyceride | | |
| | Distilled | — | 7.00 |
| | monoglycerides | | |
| 4. | Taste Modifiers-1 | | |
| | Flavor - Mint,/Fruit/ | 1.00 | 1.00 |
| | berry/chocolate | | |
| | Powder | | |
| | Potent sweetener- | 0.35 | 0.35 |
| | sucralose,/ | | |
| | aspartame- | | |
| | acesulfame k | | |
| 5. | Liquid binding | | |
| | systems | | |
| | Maltitol syrup | 21.00 | 19.00 |
| | Polysaccharide/gums | 0.500 | 0.50 |
| 6. | Coloring agent | 0.10 | 0.10 |
| | Total | 93.225 | 94.25 |

Part A Process: Prepare granules as per the process sheet.

### Part B

### Resilient Granules Composition

### Dry Blending System

| | | Example | Example |
|---|---|---|---|
| | | B.1. | B.2 |
| S. N | Materials by functionality | Percentage | Percentage |
| 1. | Lubricating agents | | |
| | Magnesium stearate | 1.00 | 1.50 |
| | Talc | 0.500 | 0.50 |
| | Colloidal silicon dioxide | 3.00 | 2.50 |
| | Hydrogenated Fat/ | — | — |
| | emulsifier/wax | | |
| 2. | Taste Modifiers-2 | | |
| | Flavor - | 2.00 | 1.00 |
| | mint/fruit/berry/chocolate | | |
| | Sweetener- | 0.125 | 0.10 |
| | sucralose/aspartame- | | |
| | acesulfame k | | |
| 3. | Coloring agent | 0.15 | 0.15 |
| | Total | 6.775 | 5.75 |

Final blending: Mix part a resilient granules with lubricating agents part in a Hobart mixer

Part C: Final product forming/compression:

The final blend of granules was compressed on a compression machine using oval or round shaped punches.

### Compression Tests

| S. N | Tests | Results | |
|---|---|---|---|
| 1. | Granules Flow in | Very good | |
| | feed frames and | | |
| | tabletting machine | | |
| 2. | Tablet Ejection | Freely | No sticking |
| | from die | ejected | observed. |
| 2. | Weight | 1.00 grams | No weight |
| | | | variation was |
| | | | observed. |
| | | | Relative |
| | | | standard |
| | | | deviation is less |
| | | | than 3.00% |
| 3. | Tablet hardness | 3 to 5 kilo | |
| | | ponds | |
| 4. | Friability | Less than | |
| | | 0.3% | |
| 5. | Tablet breakage- | No | |
| | reconnection. | powder. | |
| | Tablet was broken | Easily | |
| | with light pressure | stuck back | |
| | from fingers and | as original | |
| | reconnected. | tablet | |

Part D: Coating. This step is optional. The product was film color coated with polymer solvent color

Part D: Coating

### Example 21

### Pain Medication

### Hydrocodone and Ibuprofen

Objective: Sugared chewable tablet product.

### Part A

### Resilient Granules Composition

| | | Example | |
|---|---|---|---|
| | | 1.A.1 | |
| | | % Sugared | Example |
| | Materials by | product | 1.A.2 |
| S. N | functionality | with drug | % Sugar |
| 1. | Active ingredient | | |
| | Hydrocodone | 1.00 | — |
| | Ibuprofen or powder | 20.00 | — |
| | or granulated | | |
| 1. | Diluents/fillers | | |
| | Maltodextrin/corn | 15.00 | 15.00 |
| | syrup solids | | |
| | Saccharide/Sugar | 18.45 | 14.35 |
| | Polyols/Maltitol/ | — | — |
| | sorbitol/isomalt | | |
| 2. | Binding/Salivating/ | | |
| | Lubricating agents | | |
| | Mono and | 8.00 | 8.00 |
| | diglycerides | | |
| | Polyethylene glycol | 4.00 | — |
| | 8000 | | |
| | Hydrogenated soy | 3.00 | — |
| | bean/cottonseed oil | | |
| | Acetylated | 4.00 | 3.00 |
| | monoglyceride | | |
| | Distilled | — | 7.00 |
| | monoglycerides | | |
| 3. | Taste Modifiers-1 | | |
| | Flavor - Mint,/Fruit/ | 1.50 | 2.00 |
| | berry/chocolate | | |
| | Powder | | |
| | Potent sweetener- | 0.25 | 0.25 |
| | sucralose,/ | | |
| | aspartame- | | |
| | acesulfame k | | |
| 4. | Liquid binding | | |
| | systems | | |
| | Maltitol syrup | 19.50 | 17.00 |
| | Polysaccharide/ | 0.50 | 0.50 |
| | Gums | | |
| 5. | Coloring agent | 0.10 | 0.10 |
| | Total | 95.30 | 60.00 |

Part A Process: Prepare granules as per the process sheet.

### Part B

### Resilient Granules Composition Dry Blend with and without Actives System

| | | Example | |
|---|---|---|---|
| | | 1.B.1. | Example 1.B.2 |
| S. N | Materials by functionality | Percentage | Percentage |
| 1. | Active Ingredients | | |
| | Hydrocodone | — | 0.66 |
| | Ibuprofen powder or | — | 20.00 |
| | granulated | | |
| 2. | Diluents/fillers/dry | | |
| | binding agents | | |
| | Maltodextrin/corn syrup | — | — |
| | solids | | |
| | Dextrose/sugar/polyol | — | 13.36 |
| 3. | Lubricating agents | | |
| | Magnesium stearate | 1.00 | 1.50 |
| | Talc | 0.500 | 0.50 |
| | Silicon dioxide | 2.00 | 2.50 |
| | Hydrogenated Fat/ | — | — |
| | emulsifier/wax | | |
| 4. | Taste Modifiers-1 | | |
| | Flavor - | 1.00 | 1.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.1 | 0.1 |
| | Total | 4.70 | 40.00 |

Part B process: Blend part A granules with part B blend in a blender or Hobart mixer. The total blend is ready for compression

Part C: Final product forming

1. Compression:

The final blend of granules was compressed on a compression machine using round beveled edge punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product is made on specially designed cut/wrap machine for inserting stick for lollipop.

Part D: Coating. This step is optional. The product was film color coated with polymer solvent color. Product was also coated with sugar coating.

### Example 22

### Pain Medication

### Hydrocodone and Acetaminophen

Objective: Sugared chewable tablet product.

### Part A

### Resilient Granules Composition

| | | Example | |
|---|---|---|---|
| | | 1.A.1 | |
| | | % Sugared | Example |
| | Materials by | product | 1.A.2 |
| S. N | functionality | with drug | % Sugar |
| 1. | Active ingredient | | |
| | Hydrocodone | 0.66 | — |
| | Acetaminophen | 21.66 | — |
| 1. | Diluents/fillers/dry | | |
| | binding agents | | |
| | Maltodextrin/corn | 15.00 | 15.00 |
| | syrup solids | | |
| | Saccharide/Sugar | 17.20 | 7.15 |
| | Polyols/Maltitol/ | — | |
| | sorbitol/isomalt | | |
| 2. | Binding/Salivating/ | | |
| | Lubricating agents - | | |
| | emulsifiers/fats/waxes | | |
| | Mono and | 8.00 | 8.00 |
| | diglycerides | | |
| | Polyethylene glycol | 4.00 | — |
| | 8000 | | |
| | Hydrogenated soy | 3.00 | — |
| | bean/cottonseed oil | | |
| | Acetylated | 4.00 | 3.00 |
| | monoglyceride | | |
| | Distilled | — | 7.00 |
| | monoglycerides | | |
| 3. | Taste Modifiers-1 | | |
| | Flavor - Mint,/Fruit/ | 1.50 | 2.00 |
| | berry/chocolate | | |
| | Powder | | |
| | Potent sweetener- | 0.25 | 0.25 |
| | sucralose,/ | | |
| | aspartame- | | |
| | acesulfame k | | |
| | Souring agent-citric | — | — |
| | acid/ | | |
| 4. | Liquid binding | | |
| | systems | | |
| | Maltitol syrup | 19.50 | 17.00 |
| | Corn syrup 44 be | — | — |
| | Polysaccharide/ | 0.50 | 0.50 |
| | Gums | | |
| 5. | Coloring agent | 0.10 | 0.10 |
| | Total | 95.30 | 60.00 |

Part A Process: Prepare granules as per the process sheet.

### Part B

### Resilient Granules Composition Dry Blend with and without Actives System

| | | Example | |
|---|---|---|---|
| | | 1.B.1. | Example 1.B.2 |
| S. N | Materials by functionality | Percentage | Percentage |
| 1. | Active Ingredients | | |
| | Hydrocodone | — | 0.66 |
| | Acetaminophen | — | 21.66 |
| 2. | Diluents/fillers/dry | | |
| | binding agents | | |
| | Maltodextrin/corn syrup | — | — |
| | solids | | |
| | Dextrose/sugar/polyol | — | 11.98 |
| 3. | Lubricating agents | | |
| | Magnesium stearate | 1.00 | 1.50 |
| | Talc | 0.500 | 0.50 |
| | Silicon dioxide | 2.00 | 2.50 |
| | Hydrogenated Fat/ | — | — |
| | emulsifier/wax | | |
| 4. | Taste Modifiers-1 | | |
| | Flavor - | 1.00 | 1.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.1 | 0.1 |
| | Total | 4.70 | 40.00 |

Part B process: Blend part A granules with part B blend in a blender or Hobart mixer. The total blend is ready for compression

Part C: Final product forming

1. Compression:

The final blend of granules were compressed on a compression machine using round beveled edge punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product is made on specially designed cut/wrap machine for inserting stick for lollipop.

Part D: Coating. This step is optional. The product was film color coated with polymer solvent color. Product was also coated with sugar coating.

### Example 23

### Pain Medication

### Oxycodone and Acetaminophen

Objective: Sugared chewable tablet product.

### Part A

### Resilient Granules Composition

| | | Example | |
|---|---|---|---|
| | | 1.A.1 | |
| | | % Sugared | Example |
| | Materials by | product | 1.A.2 |
| S. N | functionality | with drug | % Sugar |
| 1. | Active ingredient | | |
| | Oxycodone | 0.33 | — |
| | Acetaminophen | 21.66 | — |
| 1. | Diluents/fillers/dry | | |
| | binding agents | | |
| | Maltodextrin/corn | 15.00 | 15.00 |
| | syrup solids | | |
| | Saccharide/Sugar | 17.55 | 7.15 |
| | Polyols/Maltitol/ | — | |
| | sorbitol/isomalt | | |
| 2. | Binding/Salivating/ | | |
| | Lubricating agents - | | |
| | waxes/fats/waxes | | |
| | Mono and | 8.00 | 8.00 |
| | diglycerides | | |
| | Polyethylene glycol | 4.00 | — |
| | 8000 | | |
| | Hydrogenated soy | 3.00 | — |
| | bean/cottonseed oil | | |
| | Acetylated | 4.00 | 3.00 |
| | monoglyceride | | |
| | Distilled | — | 7.00 |
| | monoglycerides | | |
| 3. | Taste Modifiers-1 | | |
| | Flavor - Mint,/Fruit/ | 1.50 | 2.00 |
| | berry/chocolate | | |
| | Powder | | |
| | Potent sweetener- | 0.25 | 0.25 |
| | sucralose,/ | | |
| | aspartame- | | |
| | acesulfame k | | |
| | Souring agent-citric | — | — |
| | acid/ | | |
| 4. | Liquid binding | | |
| | systems | | |
| | Maltitol syrup | 19.50 | 17.00 |
| | Corn syrup 44 be | — | — |
| | Polysaccharide/ | 0.50 | 0.50 |
| | Gums | | |
| 5. | Coloring agent | 0.10 | 0.10 |
| | Total | 95.30 | 60.00 |

Part A Process: Prepare granules as per the process sheet.

### Part B

### Resilient Granules Composition Dry Blend with and without Actives System

| | | Example | |
|---|---|---|---|
| | | 1.B.1. | Example 1.B.2 |
| S. N | Materials by functionality | Percentage | Percentage |
| 1. | Active Ingredients | | |
| | Hydrocodone | — | 0.33 |
| | Acetaminophen | — | 21.66 |
| 2. | Diluents/fillers/dry | | |
| | binding agents | | |
| | Maltodextrin/corn syrup | — | — |
| | solids | | |
| | Dextrose/sugar/polyol | — | 12.31 |
| 3. | Lubricating agents | | |
| | Magnesium stearate | 1.00 | 1.50 |
| | Talc | 0.500 | 0.50 |
| | Silicon dioxide | 2.00 | 2.50 |
| | Hydrogenated Fat/ | — | — |
| | emulsifier/wax | | |
| 4. | Taste Modifiers-1 | | |
| | Flavor - | 1.00 | 1.00 |
| | mint/fruit/berry/chocolate | | |
| | Sweetener- | 0.10 | 0.10 |
| | sucralose/aspartame- | | |
| | acesulfame k | | |
| | Souring agent-citric acid | — | — |
| 5. | Coloring agent | 0.1 | 0.1 |
| | Total | 4.70 | 40.00 |

Part B process: Blend part A granules with part B blend in a blender or Hobart mixer. The total blend was ready for compression

Part C: Final product forming

1. Compression:

The final blend of granules was compressed on a compression machine using round beveled edge punches.

### Compression Tests

| S. N | Tests | Results | |
|---|---|---|---|
| 1. | Granules Flow | Very good. No | |
| | observation in tabletting | problem was | |
| | machine | observed | |
| 2. | Piece Weight: weight | Good. | No weight |
| | varies depending upon the | | variation was |
| | drug dosage. | | observed |
| | 1.0 gram weight was | | |
| | targeted. | | |
| 3. | Tablet hardness | 5 to 7 kilo ponds | |
| 4. | Friability | Less than 0.2% | |
| 5. | Tablet breakage- | No powder. Easily | |
| | reconnection | stuck back as | |
| | | original tablet | |

2. Product is made on specially designed cut/wrap machine for inserting stick for lollipop.

Part D: Coating. This step is optional. The product was film color coated with polymer solvent color. Product was also coated with sugar coating.

### Example 24

### Zolpidem Tartrate

The compositions in the following Examples were prepared in accordance with the following flow chart.

Process Flow Sheet
Product: Rapid Melts/Regular granules

| STAGE | OPERATION | PROCESS | EQUIPMENT |
|---|---|---|---|
| Step 1: | Weighing/ Dispensing | 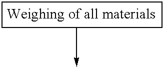 | 1. Weighing balance. 2. scoops. 3. Containers |
| Step 2: Step 3: | Sifting Blending | 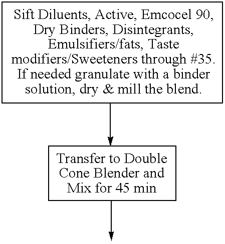 | 1. Sifter 2. 16 Screen 3. 34 Screen 1. Double Cone Blender. |
| Step 4: Step 5: | Lubrication Compression | 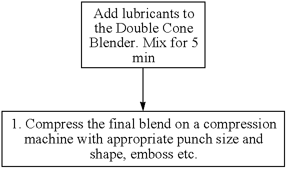 | 1. Sifter 2. Screen #34 3. Double Cone Blender 1. Tabletpress 2. Deduster 3. Punchsets |

Part A: Resilient granules, Part B: Non resilient regular granules Part C: Lubrication Composition with actives into a single layer tablet.

| S. No. | Material Name | Qty. % |
|---|---|---|
| | PART A | |
| | Active Ingredient | |
| | Zolpidem Tartrate | 2.33 |
| | Binding/Salivating/Lubricating agents - | |
| | emulsifiers/fats/waxes | |
| | Mono and Diglycerides | 3.00 |
| | PEG 3350 Granular NF | 2.25 |
| | Acetylated Monoglycerides | 3.00 |
| | Distilled Monoglycerides | 1.50 |
| | Diluents/fillers/dry binding agents/Base | |
| | Maltodextrin | 8.55 |
| | Sugar | 15.37 |
| | Liquid Binding System | |
| | Maltitol Syrup | 10.00 |
| | Sub Total | 46.00 |
| | PART-B | |
| | Active Ingredient | |
| | Zolpidem Tartrate | 2.73 |
| | Diluents/fillers/dry binding agents | |
| | Mannitol Granules | 29.66 |
| | Microcrystalline Cellulose | 12.90 |
| | Povidone | 0.37 |
| | Emulsifiers | |
| | Polysorbate 80 | 0.13 |
| | Sodium Lauryl Sulfate | 0.03 |
| | Sorbitan Monostearate | 0.50 |
| | Sodium Starch Glycolate | 1.72 |
| | Crospovidone | 2.00 |
| | Sub Total | 50.00 |
| | PART-C Lubrication | |
| | Magnesium Stearate | 1.05 |
| | Talc | 1.42 |
| | Silicon dioxide | 1.53 |
| | Sub Total | 4.00 |
| | Total | 100.00 |
| | Tablet weight (mg) | 197.63 |

Part A Process: Prepare granules as per resilient granules process sheet.

Part B: Process: Prepare granules as per rapid melts/regular granules process sheet.

Part C process: Blend part A granules with part B granules in Hobart bowl mixer and finally lubricate it.

Part D: Final product forming/compression:

Blend was compressed using oval shaped or round beveled edged punches. The results are set out below

Part E: Final product forming/compression:

The final blend of granules were compressed on a compression machine using oval or round shaped punches.

### Compression Tests

| S. N | Tests | Results | |
|---|---|---|---|
| 1. | Granules Flow observation in | Very good | |
| | machine | | |
| 2. | Weight | 2.50 grams | No weight variation |
| | | | was observed |
| 3. | Tablet hardness | 5 to 7 kiloponds | |
| 4. | Friability | Less than 0.5% | |
| 5. | Tablet breakage-reconnection. | No powder. Easily stuck | Formed into |
| | Break the tablet into two pieces | back as original tablet | original tablet |
| | with fingers and reconnect. | | |
| 6. | Compression force = 15-40 kN | | |

Part F: Coating. This step is optional. The product was film color coated with polymer solvent color.

### Example 25

### Alendronate Sodium

Part A: Resilient granules, Part B: Non resilient regular granules.

### Composition with Actives into a Bi-Layer Tablet

| S. No. | Material Name | Qty. % |
|---|---|---|
| | PART A | |
| | Active Ingredient | |
| | Alendronate Sodium | 2.33 |
| | Binding/Salivating/Lubricating agents | |
| | emulsifiers/fats/waxes | |
| | Mono and Diglycerides | 3.00 |
| | PEG 3350 Granular NF | 2.25 |
| | Acetylated Monoglycerides | 3.00 |
| | Distilled Monoglycerides | 1.50 |
| | Diluents/fillers/dry binding agents/Base | |
| | Maltodextrin | 8.54 |
| | Sugar | 16.58 |
| | Liquid Binding System | |
| | Maltitol Syrup | 10.00 |
| | Coloring agent | |
| | FD &C Yellow #6 | 0.05 |
| | Lubrication | |
| | Magnesium Stearate | 0.75 |
| | Talc | 0.50 |
| | Silicon dioxide | 1.50 |
| | Sub Total | 50.00 |
| | PART-B | |
| | Active Ingredient | |
| | Alendronate Sodium | 2.73 |
| | Diluents/fillers/dry binding agents | |
| | Mannitol Granules | 29.66 |
| | Microcrystalline Cellulose | 12.43 |
| | Povidone | 0.37 |
| | Emulsifiers | |
| | Polysorbate 80 | 0.13 |
| | Sodium Lauryl Sulfate | 0.03 |
| | Sorbitan Monostearate | 0.50 |
| | Disintegrants | |
| | Sodium Starch Glycolate | 1.70 |
| | Crospovidone | 2.00 |
| | Lubrication | |
| | Magnesium Stearate | 0.3 |
| | Talc | 0.1 |
| | Silicon dioxide | 0.05 |
| | Sub Total | 50.00 |
| | Total | 100.00 |
| | Tablet weight (mg) | 1032.00 |

Part A Process: Prepare granules as per resilient granules process sheet.

Part B: Process: Prepare granules as per rapid melts/regular granules process sheet.

Part C: Final product forming/compression:

Blend is compressed on bi-layer tablet press using a oval shaped or round beveled edged punches. The results are set out below:

Part D: Final product forming/compression:

The final blend of granules was compressed on a bi-layer tablet press using oval or round shaped punches.

### Compression Tests

| S. N | Tests | Results | |
|---|---|---|---|
| 1. | Granules Flow observation in | Very good | |
| | machine | | |
| 2. | Weight | 2.50 grams | No weight variation |
| | | | was observed |
| 3. | Tablet hardness | 5 to 7 kiloponds | |
| 4. | Friability | Less than 0.5% | |
| 5. | Tablet breakage-reconnection. | No powder. Easily stuck | Formed into |
| | Break the tablet into two pieces | back as original tablet | original tablet |
| | with fingers and reconnect. | | |
| 6. | Compression force = 15-40 kN | | |

Part E: Coating. This step is optional. The product was film color coated with polymer solvent color.

### Example 26

### Alendronate Sodium

Part A: Resilient granules Immediate Release, Part B: Resilient granules Extended Release.

### Composition with Actives into a Bi-Layer Tablet

| S. No. | Material Name | Qty. % |
|---|---|---|
| | PART A - Immediate Release | |
| | Active Ingredient | |
| | Alendronate Sodium | 2.33 |
| | Binding/Salivating/Lubricating agents - | |
| | emulsifiers/fats/waxes | |
| | Mono and Diglycerides | 3.00 |
| | PEG 3350 Granular NF | 2.25 |
| | Acetylated Monoglycerides | 3.00 |
| | Distilled Monoglycerides | 1.50 |
| | Diluents/fillers/dry binding agents/Base | |
| | Maltodextrin | 10.77 |
| | Sugar | 14.25 |
| | Liquid Binding System | |
| | Maltitol Syrup | 10.00 |
| | Coloring agent | 0.15 |
| | Lubrication | |
| | Magnesium Stearate | 0.75 |
| | Talc | 0.50 |
| | Silicon dioxide | 1.50 |
| | Sub Total | 50.00 |
| | PART-B Extended Release | |
| | Active Ingredient | |
| | Alendronate Sodium | 2.33 |
| | Binding/Salivating/Lubricating agents - | |
| | emulsifiers/fats/waxes | |
| | Mono and Diglycerides | 3.00 |
| | PEG 3350 Granular NF | 1.00 |
| | Acetylated Monoglycerides | 3.00 |
| | Distilled Monoglycerides | 1.50 |
| | Diluents/fillers/dry binding agents/Base | |
| | Maltodextrin | 8.54 |
| | Sugar | 6.50 |
| | Liquid Binding System | |
| | Maltitol Syrup | 7.00 |
| | Release Modifiers | |
| | Hypromellose | 11.38 |
| | Hydroxypropyl Cellulose | 3.00 |
| | Lubrication | |
| | Magnesium Stearate | 0.75 |
| | Talc | 0.50 |
| | Silicon dioxide | 1.50 |
| | Sub Total | 50.00 |
| | Total | 100.00 |
| | Tablet weight (mg) | 1120.00 |

Part A Process: Prepare granules as per resilient granules process sheet.

Part B: Process: Prepare granules as per resilient granules process sheet.

Part C: Final product forming/compression:

Blend is compressed on bi-layer tablet press using oval shaped or round beveled edged punches. The results are set out below.

Part D: Final product forming/compression:

The final blend of granules Blend was compressed on bi-layer tablet press using oval or round shaped punches.

### Compression Tests

| S. N | Tests | Results | |
|---|---|---|---|
| 1. | Granules Flow observation in | Very good | |
| | machine | | |
| 2. | Weight | 2.50 grams | No weight variation |
| | | | was observed |
| 3. | Tablet hardness | 5 to 7 kiloponds | |
| 4. | Friability | Less than 0.5% | |
| 5. | Tablet breakage-reconnection. | No powder. Easily stuck | Formed into |
| | Break the tablet into two pieces | back as original tablet | original tablet |
| | with fingers and reconnect. | | |
| 6. | Compression force = 15-40 kN | | |

Part E: Coating. This step is optional. The product was film color coated with polymer solvent color

### Example 27

### Lovastatin 20.0 mg+Niacin

### Example 4

### Part A: Resilient granules Immediate Release, Part B: Resilient granules Extended Release, Different active drugs.

### Composition with Actives into a Bi-Layer Tablet

| S. No. | Material Name | Qty. % |
|---|---|---|
| | PART A - Immediate Release | |
| | Active Ingredient | |
| | Lovastatin | 1.49 |
| | Binding/Salivating/Lubricating agents - | |
| | emulsifiers/fats/waxes | |
| | Mono and Diglycerides | 1.34 |
| | PEG 3350 Granular NF | 1.01 |
| | Acetylated Monoglycerides | 1.34 |
| | Distilled Monoglycerides | 0.67 |
| | Diluents/fillers/dry binding agents/Base | |
| | Maltodextrin | 4.41 |
| | Sugar | 6.37 |
| | Liquid Binding System | |
| | Maltitol Syrup | 4.47 |
| | Coloring agent | 0.02 |
| | Lubrication | |
| | Magnesium Stearate | 0.34 |
| | Talc | 0.22 |
| | Silicon dioxide | 0.67 |
| | Sub Total | 22.35 |
| | Weight of this IR layer (mg) | 300.30 |
| | PART-B Extended Release | |
| | Active Ingredient | |
| | Niacin | 37.21 |
| | Binding/Salivating/Lubricating agents - | |
| | emulsifiers/fats/waxes | |
| | Mono and Diglycerides | 4.66 |
| | Distilled Monoglycerides | 2.33 |
| | Diluents/fillers/dry binding agents/Base | |
| | Maltodextrin | 7.05 |
| | Liquid Binding System | |
| | Maltitol Syrup | 15.53 |
| | Release Modifiers | |
| | PEG 8000 Granular NF | 1.55 |
| | Hypromellose | 1.94 |
| | Hydroxypropyl Cellulose | 3.11 |
| | Lubrication | |
| | Magnesium Stearate | 1.16 |
| | Talc | 0.78 |
| | Silicon dioxide | 2.33 |
| | Sub Total | 77.65 |
| | Weight of this IR layer (mg) | 1043.40 |
| | Tablet weight (mg) | 1343.70 |

Part A Process: Prepare granules as per resilient granules process sheet.

Part B: Process: Prepare granules as per resilient granules process sheet.

Part C: Final product forming/compression:

Blend is compressed on bi-layer tablet press using oval shaped or round beveled edged punches. The results are set out below

Part D: Final product forming/compression:

The final blend of granules blend is compressed on bi-layer tablet press using oval or round shaped punches.

### Compression Tests

| S. N | Tests | Results | |
|---|---|---|---|
| 1. | Granules Flow observation in | Very good | |
| | machine | | |
| 2. | Weight | 2.50 grams | No weight variation |
| | | | was observed |
| 3. | Tablet hardness | 5 to 7 kiloponds | |
| 4. | Friability | Less than 0.5% | |
| 5. | Tablet breakage-reconnection. | No powder. Easily stuck | Formed into |
| | Break the tablet into two pieces | back as original tablet | original tablet |
| | with fingers and reconnect. | | |
| 6. | Compression force = 15-40 kN | | |

Part E: Coating. This step is optional. The product was film color coated with polymer solvent color.

### Example 28

### Atorvastatin 40.0 mg+Nifedipine30 mg

Part A: Resilient granules Immediate Release, Part B: Non-resilient regular granules Extended Release, Different active drugs.

### Composition with Actives into a Bi-Layer Tablet

| S. No. | Material Name | Qty. % |
|---|---|---|
| | PART A - Immediate Release | |
| | Active Ingredient | |
| | Atorvastatin Calcium | 5.98 |
| | Binding/Salivating/Lubricating agents - | |
| | emulsifiers/fats/waxes | |
| | Mono and Diglycerides | 5.38 |
| | PEG 3350 Granular NF | 4.05 |
| | Acetylated Monoglycerides | 5.38 |
| | Distilled Monoglycerides | 2.69 |
| | Diluents/fillers/dry binding agents/Base | |
| | Maltodextrin | 17.69 |
| | Sugar | 25.55 |
| | Liquid Binding System | |
| | Maltitol Syrup | 17.93 |
| | Coloring agent | 0.08 |
| | Lubrication | |
| | Magnesium Stearate | 1.36 |
| | Talc | 0.88 |
| | Silicon dioxide | 2.69 |
| | Sub Total | 89.66 |
| | Weight of this IR layer (mg) | 1300.00 |
| | PART - B Extended Release | |
| | Active | |
| | Nifedipine | 2.07 |
| | Diluents | |
| | Microcrystalline cellulose Powder | 0.83 |
| | Lactose | 2.61 |
| | Osmotic agent | |
| | Sodium chloride | 0.10 |
| | Release Controlling Parameters | |
| | HPMC K100LV | 3.27 |
| | HPMC K4 M | 0.93 |
| | Granulation | |
| | Povidone K30 | 0.43 |
| | Lubrication | |
| | Magnesium stearate | 0.10 |
| | Sub Total | 10.34 |
| | Weight of this IR layer (mg) | 150.00 |
| | Tablet weight (mg) | 1450.00 |

Part A Process: Prepare granules as per resilient granules process sheet.

Part B: Process: Prepare granules as per rapid melts/regular granules process sheet.

Part C: Final product forming/compression: Blend is compressed on bi-layer tablet press using a oval shaped or round beveled edged punches. The results are set out below.

Part D: Final product forming/compression:

The final blend of granules is compressed on a bi-layer tablet press using oval or round shaped punches.

### Compression Tests

| S. N | Tests | Results | |
|---|---|---|---|
| 1. | Granules Flow observation in | Very good | |
| | machine | | |
| 2. | Weight | 2.50 grams | No weight variation |
| | | | was observed |
| 3. | Tablet hardness | 5 to 7 kiloponds | |
| 4. | Friability | Less than 0.5% | |
| 5. | Tablet breakage-reconnection. | No powder. Easily stuck | Formed into |
| | Break the tablet into two pieces | back as original tablet | original tablet |
| | with fingers and reconnect. | | |
| 6. | Compression force = 15-40 kN | | |

Part E: Coating. This step is optional. The product was film color coated with polymer solvent color.

### Example 29

### 1. Procedure for Testing of Uncoated Zinc Flextabs

### Cinnamon Flavor

1.1 Average Weight:
1.1.1 Accurately weigh 20 uncoated chew melts and record the weight.

$$Average weight = \frac{Weight of 20 chew melts}{20}$$

$$Limit \text{:} 1.000 g \pm 5 \% .$$

1.2 Friability: (Ref: USP/NF<1216>)
1.2.1 Take a sample of ten whole chew melts. Accurately weigh the chew melt sample, and place it in the drum. Rotate the drum 100 times, and remove the chew melts. Remove any loose dust from the sample as before, and accurately weigh. Calculate the percentage loss of weight.

1.3 Assay:
1.3.1 Procedure: Cut 10 chew melts to small pieces and prepare a composite mixture. Accurately weigh about 1 g of sample, dissolve in about 100 mL of USP water in a 250 mL conical flask and heat until dissolved. Add about 0.2 g of activated charcoal and mix for about 5 minutes. Filter the solution through a medium porosity (25 μm) filter paper. Wash the filter paper with 2×25 mL water. Take the filtrate in a 250 mL conical flask, add 5 mL of ammonia-ammonium chloride buffer TS and 0.5 mL of eriochrome black TS, and titrate with 0.01 M disodium ethylenediaminetetraacetate VS until the solution is deep blue in color. Each mL of 0.01 M disodium ethylenediaminetetraacetate is equivalent to 0.6536 mg of elemental Zinc.

$$\frac{Titer value \times 0.6536 \times Molarity of EDTA \times Ave . weight ( mg ) \times 100}{0.01 ⨯ label claim ( mg ) ⨯ weight of sample ( mg )}$$

$$Limit \text{:} Not less than 10.0 mg and not more than 12.0 mg of elemental Zinc .$$

1.4 Content Uniformity:
1.4.1 Assay 10 chew melts individually. Accurately weigh one chew melt and cut it into small pieces. Transfer the chew melt to a 250 mL conical flask, add about 100 mL of USP water and heat until dissolved. Add about 0.2 g of activated charcoal and mix for about 5 minutes. Filter the solution through a medium porosity (25 μm) filter paper. Wash the filter paper with 25 mL water. Take the filtrate in a 250 mL conical flask, add 5 mL of ammonia-ammonium chloride buffer TS and 0.5 mL of eriochrome black TS, and titrate with 0.01 M disodium ethylenediaminetetraacetate VS until the solution is deep blue in color. Each mL of 0.01 M disodium ethylenediaminetetraacetate is equivalent to 0.6536 mg of elemental Zinc.

Calculation:

$$\frac{Titer value \times 0.6536 \times Molarity of EDTA \times 100}{0.01 \times 10.5}$$

$$Acceptance criteria \text{:} 1. Assay \text{:} Not less than 85.0 \% and not more than 115.0 \%$$

$$2. RSD \leq 6.0 \%$$

### Example 30

### 2. Procedure for Testing of Coated Zicam Flextabs

### Cinnamon Flavor

2.1 Description:

Zicam Chew Melts—Cinnamon Flavor are coated and glazed red color arc rectangle shaped tablets.

2.2 Average Weight:
2.2.1 Accurately weigh 20 coated chew melts and record the weight.

$$Average weight = \frac{Weight of 20 chew melts}{20}$$

$$Limit \text{:} 1.005 g \pm 5 \% .$$

2.3 Water Activity:
2.3.1 Procedure: Place 1 chew in the sample holder and cover with the probe over it. Simultaneously press “start” button on probe 1 of AW measurement window and Red button on the probe. Note down the values of the water activity and the temperature as alarm buzzes at the completion of measurement. Test 5 Chews and record average water activity value.
2.3.2 Limit: NMT 1.0 AW.

2.4 Flavor/Taste:

Characteristic cinnamon flavor and taste.

Name of the product: Zinc Flextabs—Cinnamon flavor

Batch#: CPI-139-CC-460

Assay=94.8%

Weight Variation=1.2%

Friability=0.2%

### Example 31

Name of the product: Zinc Flextabs—Cinnamon Flavor

Stability studies under Accelerated conditions (40° C./75% RH)

Mfg. Date: JUL 06

Packing Details: 25 ChewCaps packed in a 60 cc HDPE bottle, with heat seal, containing non-adsorbent cotton and desiccant silica bag.

| | | | | 1 M | 2 M | 3 M | 4 M | 5 M | 6 M |
|---|---|---|---|---|---|---|---|---|---|
| | | | | 18 JAN | 18 FEB | 18 MAR | 18 APR | 18 MAY | 18 JUN |
| No | Tests | Specifications | Initial 15 DEC 06 | 07 | 07 | 07 | 07 | 07 | 07 |
| 1. | Description | Zinc | CONFORMS | CONFORMS | Shiny | Shiny | Shiny | Shiny red | Shiny red |
| | | Flextabs - | | | red | red | red | tablets, | tablets, |
| | | Cinnamon | | | tablets, | tablets, | tablets, | Conforms | Conforms |
| | | Flavor are | | | Conforms | Conforms | Conforms | | |
| | | shiny red | | | | | | | |
| | | arc | | | | | | | |
| | | rectangle | | | | | | | |
| | | shaped | | | | | | | |
| | | chew tabs | | | | | | | |
| 2. | Ave. wt. | NLT 1.0 g | 1.0 g | 1.0 g | 1.0 g | 1.0 g | 1.0 g | 1.0 g | 1.0 g |
| 3. | Assay | NLT 10.0 mg | 11.0 mg | 10.6 mg | 11.0 mg | 10.9 mg | 9.6 mg | 10.7 mg | 11.1 mg |
| | | and | | | | | | | |
| | | NMT 12.0 mg | | | | | | | |
| | | of | | | | | | | |
| | | elemental | | | | | | | |
| | | Zinc | | | | | | | |
| 4. | Water | NMT 1.0 | 0.4 AW | 0.3 AW | 0.2 AW | 0.2 AW | 0.3 AW | 0.5 AW | 0.5 AW |
| | Activity | AW | | | | | | | |
| 5. | Taste & | Must | CONFORMS | CONFORMS | CONFORMS | CONFORMS | CONFORMS | CONFORMS | Conforms |
| | Mouth | comply with | | | | | | | |
| | Feeling | control | | | | | | | |
| | | sample | | | | | | | |
| 6. | Flavor | Must | CONFORMS | CONFORMS | CONFORMS | CONFORMS | CONFORMS | CONFORMS | Conforms |
| | | comply with | | | | | | | |
| | | control | | | | | | | |
| | | sample | | | | | | | |

### Example 32

Name of the product: Zinc Flextabs—Cinnamon Flavor

Stability studies under Long term conditions (25° C./60% RH)

Mfg. Date: JUL 06

Packing Details: 25 ChewCaps packed in a 60 cc HDPE bottle, with heat seal, containing non-adsorbent cotton and desiccant silica bag.

| | | | | | | | 12^{th} | 18^{th} | 24^{th} | 36^{th} |
|---|---|---|---|---|---|---|---|---|---|---|
| | | | | 3^{rd} | 6^{th} | 9^{th} | month | month | month | month |
| | | | Initial | month | month | month | 18 | 18 | 18 | 18 |
| S. | | | 15 DEC | 18 MAR | 18 | 15 SEP | DEC | JUN | DEC | DEC |
| No | Tests | Specifications | 06 | 07 | JUN07 | 07 | 07 | 08 | 08 | 09 |
| 1. | Description | Zinc Flextabs- | Sonforms | Conforms | Conforms | | | | | |
| | | Cinnamon | | | | | | | | |
| | | Flavor are | | | | | | | | |
| | | shiny red arc | | | | | | | | |
| | | rectabgle | | | | | | | | |
| | | shaped chew | | | | | | | | |
| | | tabs | | | | | | | | |
| 2. | Avg. wt. | NLT 1.0 g | 1.0 g | 1.0 g | 1.0 g | | | | | |
| 3. | Assay | Not less than | 10.4 mg | 10.5 mg | 11.0 mg | | | | | |
| | | 10.0 mg and | | | | | | | | |
| | | Not more than | | | | | | | | |
| | | 12.0 mg of | | | | | | | | |
| | | elemental zinc | | | | | | | | |
| 4. | Water | Not more than | 0.4 AW | 0.2 AW | 0.5 AW | | | | | |
| | | 1.0 AW | | | | | | | | |
| 5. | Taste & | Must comply | Complies | Complies | Complies | | | | | |
| | Mouth | | | | | | | | | |
| | Feeling | | | | | | | | | |
| 6. | Flavor | Must comply | Complies | Complies | Complies | | | | | |
| | | with control | | | | | | | | |
| | | sample | | | | | | | | |

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

I claim:

1. A resilient self-adhering granule which comprises a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m².

2. The granule according to claim 1, wherein the polysaccharide is selected from the group consisting of simple sugars, complex sugars, fibers, starches, pectins, dextrins, dextrans, natural gums, synthetic gums, mucilages, derivatives thereof, components thereof, and mixtures thereof.

3. The granule according to claim 2, wherein the polysaccharide is a dextrin.

4. The granule according to claim 1, wherein the binder is selected from the group consisting of syrups, emulsifiers, fats, waxes, gums, plasticizers, and mixtures thereof.

5. The granule according to claim 4, wherein the binder is selected from the group consisting of maltitol syrups, acetylated mono-, di-, or triglycerides, polyethyleneglycol esters, bee's wax, carnuba wax, spermaceti, mineral oils, paraffins, microcrystalline waxes, polyethylene wax, gum Arabica, gum tragacanth, gum acacia, fiber gums, and mixtures thereof.

6. The granule according to claim 5, wherein the binder is maltitol syrup.

7. The granule according to claim 1, wherein the binder has a viscosity of about 10,000 mPa·s.

8. The granule according to claim 1, further comprising a sugar alcohol selected from the group consisting of arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, derivatives thereof, and mixtures thereof.

9. The granule according to claim 8, wherein the sugar alcohol is maltitol.

10. The granule according to claim 1, further comprising an active agent selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives including hypnotics and neuroleptics, astringents, beta-adrenoceptor blocking agents, blood products and substitutes, cardiac inotropic agents, contrast media, corticosteroids, cough suppressants including expectorants and mucolytics, diagnostic agents, diagnostic imaging agents, diuretics, dopaminergics including antiparkinsonian agents, haemostatics, immunological agents, lipid regulating agents, muscle relaxants, parasympathomimetics, parathyroid calcitonin and biphosphonates, prostaglandins, radio-pharmaceuticals, sex hormones including steroids, anti-allergic agents, stimulants and anoretics, sympathomimetics, thyroid agents, vasodilators, xanthines, antitussives, decongestants, alkaloids, laxatives, antacids, ion exchange resins, anti-cholesterolemics, antipyretics, analgesics including acetaminophen, aspirin, non-steroidal anti-inflammatory drugs and opioids, appetite suppressants, expectorants, anti-anxiety agents, anti-ulcer agents, coronary dilators, cerebral dilators, peripheral vasodilators, anti-infectives, psycho-tropics, antimanics, stimulants, gastrointestinal agents, sedatives, anti-diarrheal preparations, anti-anginal drugs, vasodilators, vasoconstrictors, migraine treatments, tranquilizers, anti-psychotics, antitumor drugs, antithrombotic drugs, hypnotics, anti-emetics, anti-nausants, anti-convulsants, neuromuscular drugs, hyper- and hypoglycemic spasmodics, uterine relaxants, antiobesity drugs, anabolic drugs, erythropoetic drugs, antiasthmatics, mucolytics, anti-uricemic drugs, and mixtures thereof.

11. The granule according to claim 10, wherein the active agent is selected from the group consisting of analgesics, antibiotics, lipid regulating agent, antihistamines, antineoplastic agents, and antiviral agents.

12. The granule, according to claim 10, wherein the active agent is homogenous throughout the granule or is coated on the surface of the granule.

13. The granule according to claim 10, wherein the active agent is in immediate release form or in controlled release form.

14. The granule of claim 1, further comprising a nutritional supplement selected from the group consisting of calcium-containing materials, stannol esters, hydroxycitric acid, vitamins, minerals, herbals, spices, and mixtures thereof.

15. The granule according to claim 13, wherein the nutritional supplement is a calcium containing material, a zinc containing material, or vitamin C.

16. The granule according to claim 1, wherein the granule withstands pressures of up to 50 kilonewtons/m² without losing its resiliency.

17. An oral dosage composition comprising resilient self-adhering granules which comprise a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m² and the granules have agglomerated to form the oral dosage composition.

18. The composition according to claim 17, wherein the polysaccharide is selected from the group consisting of simple sugars, complex sugars, fibers, starches, pectins, dextrins, dextrans, natural gums, synthetic gums, mucilages, derivatives thereof, components thereof, and mixtures thereof.

19. The composition according to claim 17, wherein the binder is selected from the group consisting of syrups, emulsifiers, fats, waxes, gums, plasticizers, and mixtures thereof.

20. The composition according to claim 17, wherein the binder has a viscosity of about 10,000 mPa·s.

21. The composition according to claim 17, further comprising a sugar alcohol selected from the group consisting of arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, derivatives thereof, and mixtures thereof.

22. The composition according to claim 17, further comprising an active agent selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives including hypnotics and neuroleptics, astringents, beta-adrenoceptor blocking agents, blood products and substitutes, cardiac inotropic agents, contrast media, corticosteroids, cough suppressants including expectorants and mucolytics, diagnostic agents, diagnostic imaging agents, diuretics, dopaminergics including antiparkinsonian agents, haemostatics, immunological agents, lipid regulating agents, muscle relaxants, parasympathomimetics, parathyroid calcitonin and biphosphonates, prostaglandins, radio-pharmaceuticals, sex hormones including steroids, anti-allergic agents, stimulants and anoretics, sympathomimetics, thyroid agents, vasodilators, xanthines, antitussives, decongestants, alkaloids, laxatives, antacids, ion exchange resins, anti-cholesterolemics, antipyretics, analgesics including acetaminophen, aspirin, non-steroidal anti-inflammatory drugs and opioids, appetite suppressants, expectorants, anti-anxiety agents, anti-ulcer agents, coronary dilators, cerebral dilators, peripheral vasodilators, anti-infectives, psycho-tropics, antimanics, stimulants, gastrointestinal agents, sedatives, anti-diarrheal preparations, anti-anginal drugs, vasodilators, vasoconstrictors, migraine treatments, tranquilizers, anti-psychotics, antitumor drugs, antithrombotic drugs, hypnotics, anti-emetics, anti-nausants, anti-convulsants, neuromuscular drugs, hyper- and hypoglycemic spasmodics, uterine relaxants, antiobesity drugs, anabolic drugs, erythropoetic drugs, antiasthmatics, mucolytics, anti-uricemic drugs, and mixtures thereof.

23. The composition according to claim 17, wherein the oral dosage composition is in the form of a tablet that has been scored at least once.

24. The composition according to claim 23, wherein the tablet is broken into two portions and the breaking results in substantially no material loss.

25. The composition according to claim 24, wherein the two broken portions may be reformed with substantially no material loss.

26. The composition according to claim 17, further comprising non-resilient granules.

27. The composition according to claim 26, wherein the resilient granules comprise an active agent and the non-resilient granules comprise an inactive agent,

28. The composition according to claim 26, wherein the resilient granules comprise an inactive agent and the non-resilient granules comprise an active agent,

29. The composition according to claim 26, wherein the resilient granules comprise an active agent in immediate release form and the non-resilient granules comprise the same active agent in delayed release form.

30. The composition according to claim 26, wherein the resilient granules comprise an active agent in delayed release form and the non-resilient granules comprise the same active agent in immediate release form.

31. The composition according to claim 26, wherein the resilient granules comprise a first active agent and the non-resilient granules comprise a second active agent, and further wherein the first and second active agents can both be in immediate release form, can both be in delayed release form, or one active agent can be in immediate release form and the other active agent can be in delayed release form.

32. The composition according to claim 17, wherein a first portion of the resilient granules comprises an active agent and a second portion of the resilient granules comprises an inactive agent.

33. The composition according to claim 17, wherein a first portion of the resilient granules comprises an active agent in immediate release form and a second portion of the resilient granules comprises the same active agent in delayed release form.

34. The composition according to claim 17, wherein a first portion of the resilient granules comprises a first active agent and a second portion of the resilient granules comprises a second active agent, and further wherein the first and second active agents can both be in immediate release form, can both be in delayed release form, or one active agent can be in immediate release form and the other active agent can be in delayed release form.

35. A method for making a resilient self-adhering granule which comprises a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m², which comprises the steps of:
(a) mixing and heating the polysaccharide and binder in a mixer to form a reaction mixture; and
(b) extruding the reaction mixture from step (a), cooling the reaction mixture to room temperature, milling the reaction mixture to a particular granule size, and cooling the reaction mixture in a freezer.

36. The method according to claim 35, wherein the polysaccharide is selected from the group consisting of simple sugars, complex sugars, fibers, starches, pectins, dextrins, dextrans, natural gums, synthetic gums, mucilages, derivatives thereof, components thereof, and mixtures thereof.

37. The method according to claim 35, wherein the binder is selected from the group consisting of syrups, emulsifiers, fats, waxes, gums, plasticizers, and mixtures thereof.

38. The method according to claim 35, wherein the binder has a viscosity of about 10,000 mPa·s.

39. The method according to claim 35, further comprising a sugar alcohol selected from the group consisting of arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, derivatives thereof, and mixtures thereof.

40. The method according to claim 35, further comprising an active agent selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives including hypnotics and neuroleptics, astringents, beta-adrenoceptor blocking agents, blood products and substitutes, cardiac inotropic agents, contrast media, corticosteroids, cough suppressants including expectorants and mucolytics, diagnostic agents, diagnostic imaging agents, diuretics, dopaminergics including antiparkinsonian agents, haemostatics, immunological agents, lipid regulating agents, muscle relaxants, parasympathomimetics, parathyroid calcitonin and biphosphonates, prostaglandins, radio-pharmaceuticals, sex hormones including steroids, anti-allergic agents, stimulants and anoretics, sympathomimetics, thyroid agents, vasodilators, xanthines, antitussives, decongestants, alkaloids, laxatives, antacids, ion exchange resins, anti-cholesterolemics, antipyretics, analgesics including acetaminophen, aspirin, non-steroidal anti-inflammatory drugs and opioids, appetite suppressants, expectorants, anti-anxiety agents, anti-ulcer agents, coronary dilators, cerebral dilators, peripheral vasodilators, anti-infectives, psycho-tropics, antimanics, stimulants, gastrointestinal agents, sedatives, anti-diarrheal preparations, anti-anginal drugs, vasodilators, vasoconstrictors, migraine treatments, tranquilizers, anti-psychotics, antitumor drugs, antithrombotic drugs, hypnotics, anti-emetics, anti-nausants, anti-convulsants, neuromuscular drugs, hyper- and hypoglycemic spasmodics, uterine relaxants, antiobesity drugs, anabolic drugs, erythropoetic drugs, antiasthmatics, mucolytics, anti-uricemic drugs, and mixtures thereof.

41. A method for administering an oral dosage composition to a subject comprising:
a) providing resilient self-adhering granules which comprises a polysaccharide present in an amount from about 10 wt % to about 90 wt % and a binder having a viscosity from about 5,000 mPa·s to about 250,000 mPa·s present in an amount from about 90 wt % to about 10 wt %, wherein the granule is capable of reversible agglomeration at or below 6,500 kilonewtons/m²; and
b) administering the oral dosage composition to a subject's oral cavity, wherein the majority of the resilient self-adhering granules is released in the gastrointestinal tract.

42. The method according to claim 41, wherein the polysaccharide is selected from the group consisting of simple sugars, complex sugars, fibers, starches, pectins, dextrins, dextrans, natural gums, synthetic gums, mucilages, derivatives thereof, components thereof, and mixtures thereof.

43. The method according to claim 41, wherein the binder is selected from the group consisting of syrups, emulsifiers, fats, waxes, gums, plasticizers, and mixtures thereof.

44. The method according to claim 41, wherein the binder has a viscosity of about 10,000 mPa·s.

45. The method according to claim 41, further comprising a sugar alcohol selected from the group consisting of arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, derivatives thereof, and mixtures thereof.

46. The method according to claim 41, further comprising an active agent selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives including hypnotics and neuroleptics, astringents, beta-adrenoceptor blocking agents, blood products and substitutes, cardiac inotropic agents, contrast media, corticosteroids, cough suppressants including expectorants and mucolytics, diagnostic agents, diagnostic imaging agents, diuretics, dopaminergics including antiparkinsonian agents, haemostatics, immunological agents, lipid regulating agents, muscle relaxants, parasympathomimetics, parathyroid calcitonin and biphosphonates, prostaglandins, radio-pharmaceuticals, sex hormones including steroids, anti-allergic agents, stimulants and anoretics, sympathomimetics, thyroid agents, vasodilators, xanthines, antitussives, decongestants, alkaloids, laxatives, antacids, ion exchange resins, anti-cholesterolemics, antipyretics, analgesics including acetaminophen, aspirin, non-steroidal anti-inflammatory drugs and opioids, appetite suppressants, expectorants, anti-anxiety agents, anti-ulcer agents, coronary dilators, cerebral dilators, peripheral vasodilators, anti-infectives, psycho-tropics, antimanics, stimulants, gastrointestinal agents, sedatives, anti-diarrheal preparations, anti-anginal drugs, vasodilators, vasoconstrictors, migraine treatments, tranquilizers, anti-psychotics, antitumor drugs, antithrombotic drugs, hypnotics, anti-emetics, anti-nausants, anti-convulsants, neuromuscular drugs, hyper- and hypoglycemic spasmodics, uterine relaxants, antiobesity drugs, anabolic drugs, erythropoetic drugs, antiasthmatics, mucolytics, anti-uricemic drugs, and mixtures thereof.

47. The method according to claim 41, further comprising non-resilient granules.

48. The method according to claim 47, wherein the resilient granules comprise an active agent and the non-resilient granules comprise an inactive agent,

49. The method according to claim 47, wherein the resilient granules comprise an inactive agent and the non-resilient granules comprise an active agent,

50. The method according to claim 47, wherein the resilient granules comprise an active agent in immediate release form and the non-resilient granules comprise the same active agent in delayed release form.

51. The method according to claim 47, wherein the resilient granules comprise an active agent in delayed release form and the non-resilient granules comprise the same active agent in immediate release form.

52. The method according to claim 47, wherein the resilient granules comprise a first active agent and the non-resilient granules comprise a second active agent, and further wherein the first and second active agents can both be in immediate release form, can both be in delayed release form, or one active agent can be in immediate release form and the other active agent can be in delayed release form.

53. The method according to claim 41, wherein a first portion of the resilient granules comprises an active agent and a second portion of the resilient granules comprises an inactive agent.

54. The method according to claim 41, wherein a first portion of the resilient granules comprises an active agent in immediate release form and a second portion of the resilient granules comprises the same active agent in delayed release form.

55. The method according to claim 41, wherein a first portion of the resilient granules comprises a first active agent and a second portion of the resilient granules comprises a second active agent, and further wherein the first and second active agents can both be in immediate release form, can both be in delayed release form, or one active agent can be in immediate release form and the other active agent can be in delayed release form.
